SEQUENCE LISTING (4 sequences)

SEQ ID NO: 1 - protein, 127 aa, synthetic construct
  REGION 1..127: note = Camelid
HVQLVESGGGSVQAGGSLRLSCAASGANGGRSCMGWFRQVPGKEREGVSGISTGGLITYYADSVKGRFTISQDNTKNTLYLQMNSLKPEDTAMYYCATSRFDCYRGSWFNRYMYNSWGQGTQVTVSS

SEQ ID NO: 2 - protein, 124 aa, synthetic construct
  REGION 1..124: note = Camelid
EVQLVESGGGSVQTGGSLRLSCAVSGNIGSSYCMGWFRQAPGKKREAVARIVRDGATGYADYVKGRFTISRDSAKNTLYLQMNRLIPEDTAIYYCAADLPPGCLTQAIWNFGYRGQGTLVTVSS

SEQ ID NO: 3 - protein, 124 aa, synthetic construct
  REGION 1..124: note = Camelid
EVQLVESGGGSVQAGGSLRLSCAASVYSYNTNCMGWFRQAPGKEREGVAVIYAAGGLTYYADSVKGRFTISQENGKNTVYLTMNRLKPEDTAMYYCAAKRWCSSWNRGEEYNYWGQGTQVTVSS

SEQ ID NO: 4 - protein, 141 aa, synthetic construct
  REGION 1..141: note = Camelid
MGDVQLVESGGDSVRAGGSLQLSCKASGYTYNSRVDIRSMGWFRQYPGKEREGVATINIRNSVTYYADSVKGRFTISQDNAKNTVYLQMNALKPEDTAMYYCALSDRFAAQVPARYGIRPSDYNYWGEGTLVTVSSSSGLE

(19) United States
(12) Patent Application Publication
(10) Pub. No.: US 2024/0101652 A1
(43) Pub. Date: Mar. 28, 2024

(54) THERAPEUTIC SINGLE DOMAIN ANTIBODY

(71) Applicant: Singh Biotechnology, LLC, Lutz, FL (US)

(72) Inventor: Sunanda Singh, Lutz, FL (US)

(73) Assignee: Singh Biotechnology, LLC, Lutz, FL (US)

(21) Appl. No.: 17/822,642

(22) Filed: Aug. 26, 2022

Related U.S. Application Data
(60) Provisional application No. 63/237,987, filed on Aug. 27, 2021.

(51) Int. Cl. C07K 16/18; A61P 35/00

(52) U.S. Cl. CPC C07K 16/18; A61P 35/00; C07K 2317/569; C07K 2317/82

(57) ABSTRACT

This invention provides compositions and methods to prevent aberrant cell proliferation in a subject using a single-domain antibody (sdAb) directed against an intracellular component, wherein the aberrant cell proliferation can be cancer. The sdAb is synergistic with one or more chemotherapeutic drugs and improves therapeutic efficacy of the one or more chemotherapeutic drug against cancer.

### CROSS-REFERENCE TO RELATED APPLICATIONS

This application claims the benefit of U.S. Provisional Patent Application No. 63/237,987, filed on Aug. 27, 2021, the contents of which are incorporated herein by reference in their entirety.

### SEQUENCE LISTING

The present application is being filed along with a Sequence Listing in electronic format. The Sequence Listing is provided as a file titled “20230412_sequence_listing.xml” created Apr. 12, 2023, and is 7,000 bytes in size. The information in the electronic format of the Sequence Listing is incorporated herein by reference in its entirety.

### BACKGROUND

Signal transducer and activator of transcription 3 (STAT3) is both a signaling molecule and a transcription factor, while the mammalian homolog of Kirsten RAS (KRAS) is a signal relaying GTP-binding protein. Both regulate cell proliferation, survival, differentiation, apoptosis, cell migration, stem cell self-renewal, inflammation and angiogenesis. Aberrant expression of STAT3 and mutant active forms of KRAS (e.g., G12D, G12C, G12V) have been well established in the induction and maintenance of multiple cancers. Due to their central role in tumor processes, STAT3 and KRAS mutant proteins have been considered anticancer targets. However, they are also considered to be clinically “undruggable” intracellular molecules due to pharmacokinetic and toxicity barriers. Inhibition of these proteins in cancer cells would reduce tumor burden and enhance clinical management of cancers; however, this critical task remains an unmet need.

Traditional chemotherapies alone are often insufficient for treatment and can result in significant toxicities, rendering many human malignancies difficult to treat, including pancreatic cancers, triple negative breast cancers (TNBC), glioblastomas, and sarcomas. In addition, new targeted therapies can often become ineffective eventually, due to development of drug resistance in cancer cells, often linked to blockade of key signaling pathways. Oncogenic mutations, loss of tumor suppressor genes, overexpression of normal proteins, or some combination of these events also contribute to drug resistance. Janus kinase (JAK)/STAT pathway is a key modulator of cellular growth, differentiation, and inflammatory response. Elevated phosphorylated STAT3 (p-STAT3) has been associated with a poor prognosis of cancers with solid tumors. Activated STAT3 forms homodimers that translocate to the nucleus, where it binds DNA to initiate the transcription of target genes associated with cellular growth, proliferation, anti-apoptosis, angiogenesis, immunosuppression, and invasion/migration. Due to its central role in tumor processes, STAT3 has been considered as a potential anticancer target since its first description as an oncogene in 1998 and has led to evaluation of STAT3 inhibitors for their antitumor activity in vitro and in vivo experimental tumor models. However, most of these inhibitors are yet to be translated to clinical use for cancer treatment, primarily due to pharmacokinetic, efficacy, and safety obstacles. KRAS mutants have been shown to be the driver mutation for ˜25% of human cancers, while most frequently present in pancreatic (98%) and colorectal (53%) cancers. The mutant form retains GTP without hydrolyzing it, thereby becoming constitutively active. Many researchers have focused on developing small molecule targets to the KRAS mutant for decades. However, problems in detecting binding pockets for these small molecules to bind KRAS have rendered this a nearly impossible task, and thus far no inhibitory drug has been approved for use in treatment.

Cancer cells utilize p-STAT3 as an escape mechanism to become resistant to chemotherapy and radiation therapy. Inhibiting STAT3 with small molecule inhibitors not only suppresses cancer growth, activates apoptosis, and inhibits angiogenesis, but it also has been shown to re-model the stroma of pancreatic cancers. Inhibiting STAT3 in human patients during Phase 1 studies have demonstrated this to be a safe and well tolerated approach. Studies have focused on identifying novel small molecule inhibitors of STAT3, which act either by inhibiting phosphorylation of STAT3, inhibiting DNA binding, or by preventing the formation of functional STAT3 dimers.

Camelid antibodies are comprised of two heavy chain immunoglobulins with one variable domain (VHH) per heavy chain. Camelid VHHs have been used to target multiple extracellular targets (e.g., IL-6R, IL-17, TNF-alpha, VWF, and others), and several VHHs are in various stages of human clinical trials (Phases II and III), with no major side effect or toxicity reported. One VHH, namely Caplacizumab, has been approved by the FDA and has been successfully commercialized for treating adult-acquired thrombotic thrombocytopenic purpura.

The present invention is directed to therapeutic uses of a 15 kDa single domain antibody (sdAb), SBT-100 (SEQ ID NO: 1). SBT-100 (SEQ ID NO: 1) binds to both STAT3 and KRAS proteins with nanomolar affinity and can penetrate into tumor cell cytosol, impair phosphorylation and nuclear translocation of STAT3, ultimately resulting in reduced VEGF levels and PD-L1 expression, decreased viral replication and significant cancer cell growth inhibition. Additionally, SBT-100 (SEQ ID NO: 1) inhibits KRAS GTPase activity and downstream phosphorylation of ERK in vitro. In addition to inhibiting growth of multiple human cancer cell lines in vitro, in athymic xenograft mouse models in both a triple negative breast cancer cell line with KRAS (G13D) mutation (MDA-MB-231) and a pancreatic cancer cell line with KRAS (G12D) mutation (PANC-1), SBT-100 (SEQ ID NO: 1) treatment reduces tumor volumes without any observable toxicity. SBT-100 (SEQ ID NO: 1) also appears unique in its ability to penetrate the blood brain barrier (BBB). These results demonstrate the feasibility of targeting hard to reach aberrant intracellular transcription factor and signaling proteins simultaneously with one VHH to improve cancer therapies.

### SUMMARY

The invention includes a method of preventing aberrant cell proliferation in a subject using a single-domain antibody (sdAb) directed against an intracellular component. In one aspect, the aberrant cell proliferation is cancer such as, for example, osteosarcoma, fibrosarcoma, glioblastoma, leukemia, pancreatic cancer, breast cancer, and prostate cancer. In another aspect, the sdAb is synergistic with one or more chemotherapeutic drugs and improves therapeutic efficacy of the one or more chemotherapeutic drug against cancer such as, for example doxorubicin and gemcitabine. In one aspect, the sbAb decreases the toxicity of one or more chemotherapeutic drugs and improves survival in the treated subject. In another aspect, the sbAb is used in combination with one or more compounds. In one aspect, intracellular component comprises a protein such as, for example STAT1, STAT2, STAT3, STAT4, STAT5a, STAT5b, or STAT6. The method of claim 1, wherein the sdAb comprises SBT-100 (SEQ ID NO: 1). A method for inhibiting the phosphorylation of STAT3, the method comprising administration of a sdAb to a patient in need thereof, such as, for example, SBT-100 (SEQ ID NO: 1).

In another embodiment, the invention includes a method for inhibiting the activation of STAT3, the method comprising administration of a sdAb to a patient in need thereof. In one aspect, the sdAb comprises SBT-100 (SEQ ID NO: 1).

In another embodiment, the invention includes a method for inhibiting T-cell proliferation, the method comprising administration of a sdAb to a patient in need thereof. In one aspect, the sdAb comprises SBT-100 (SEQ ID NO: 1).

In another embodiment, the invention includes a method for preserving vision, the method comprising administration of a sdAb to a patient in need thereof. In one aspect, the sdAb comprises SBT-100 (SEQ ID NO: 1).

In another embodiment, the invention includes a method for inhibiting the proliferation of CD4+IL-17+T cells, the method comprising administration of a sdAb to a patient in need thereof. In one aspect, the sdAb comprises SBT-100 (SEQ ID NO: 1).

In another embodiment, the invention includes a method for inhibiting disease caused by CD4+IL-17+T cells, the method comprising administration of a sdAb to a patient in need thereof. In one aspect, the sdAb comprises SBT-100 (SEQ ID NO: 1).

In another embodiment, the invention includes a method for inhibiting proliferation of CD4+IFN-gamma+T cells, the method comprising administration of a sdAb to a patient in need thereof. In one aspect, the sdAb comprises SBT-100 (SEQ ID NO: 1).

In another embodiment, the invention includes a method for inhibiting disease caused by CD4+IFN-gamma+T cell proliferation, the method comprising administration of a sdAb to a patient in need thereof. In one aspect, the sdAb comprises SBT-100 (SEQ ID NO: 1) (SEQ ID NO:1).

In another embodiment, the invention includes a method for inhibiting proliferation of CD4+IL-17+IFN-gamma+T cells, the method comprising administration of a sdAb to a patient in need thereof. In one aspect, the sdAb comprises SBT-100 (SEQ ID NO: 1) (SEQ ID NO:1).

In another embodiment, the invention includes a method for inhibiting disease caused by proliferation of CD4+IL-17+IFN-gamma+T cells, the method comprising administration of a sdAb to a patient in need thereof. In one aspect, the sdAb comprises SBT-100 (SEQ ID NO:1).

In another embodiment, the invention includes a method for inhibiting proliferation of CD4+RORgammaT+ T cells, the method comprising administration of a sdAb to a patient in need thereof. In one aspect, the sdAb comprises SBT-100 (SEQ ID NO:1).

In another embodiment, the invention includes a method for inhibiting disease caused by proliferation of CD4+RORgammaT+ T cells, the method comprising administration of a sdAb to a patient in need thereof. In one aspect, the sdAb comprises SBT-100 (SEQ ID NO:1).

In another embodiment, the invention includes a method for inhibiting proliferation of CD4+Granzyme-B+ T cells, the method comprising administration of a sdAb to a patient in need thereof. In one aspect, the sdAb comprises SBT-100 (SEQ ID NO:1).

In another embodiment, the invention includes a method for inhibiting disease caused by CD4+Granzyme-B+ T cells, the method comprising administration of a sdAb to a patient in need thereof. In one aspect, the sdAb comprises SBT-100 (SEQ ID NO:1).

In another embodiment, the invention includes a method for inhibiting proliferation of CD4+Foxp3+ T cells, the method comprising administration of a sdAb to a patient in need thereof. In one aspect, the sdAb comprises SBT-100 (SEQ ID NO:1).

In another embodiment, the invention includes a method for inhibiting proliferation of CD25+Foxp3+ T cells, the method comprising administration of a sdAb to a patient in need thereof. In one aspect, the sdAb comprises SBT-100 (SEQ ID NO:1).

In another embodiment, the invention includes a method for inhibiting disease caused by CD25+Foxp3+ T cells, the method comprising administration of a sdAb to a patient in need thereof. In one aspect, the sdAb comprises SBT-100 (SEQ ID NO:1).

In another embodiment, the invention includes a method for inhibiting proliferation of CD4+IL-10+ T cells, the method comprising administration of a sdAb to a patient in need thereof. In one aspect, the sdAb comprises SBT-100 (SEQ ID NO:1).

In another embodiment, the invention includes a method for inhibiting disease caused by CD4+IL-10+ T cells, the method comprising administration of a sdAb to a patient in need thereof. In one aspect, the sdAb comprises SBT-100 (SEQ ID NO:1).

In another embodiment, the invention includes a method for inhibiting disease caused by one or more cytokines selected from the group comprising IL-17, IFN-gamma, IL-23, GM-CSF, and IL-1alpha, the method comprising administration of a sdAb to a patient in need thereof. In one aspect, the sdAb comprises SBT-100 (SEQ ID NO:1).

In another embodiment, the invention includes a method for inhibiting proliferation of Th1, Treg and Th17 pathogenic cells, the method comprising administration of a sdAb to a patient in need thereof. In one aspect, the sdAb comprises SBT-100 (SEQ ID NO:1).

In another embodiment, the invention includes a method for inhibiting disease caused by Th1, Treg and Th17 pathogenic cells, the method comprising administration of a sdAb to a patient in need thereof. In one aspect, the sdAb comprises SBT-100 (SEQ ID NO:1). In another aspect, the disease is selected from the group comprising rheumatoid arthritis, inflammatory bowel diseases, multiple sclerosis, psoriasis, atopic dermatitis, and Type 1 diabetes mellitus.

In another embodiment, the invention includes a method for inhibiting disease caused by autoimmune diseases, the method comprising administration of a sdAb to a patient in need thereof. In one aspect, the sdAb comprises SBT-100 (SEQ ID NO:1).

In another embodiment, the invention includes a method for inhibiting VEGF production by retinal epithelial cells in an in vitro model for age-related macular degeneration (AMD), the method comprising administration of a sdAb to a patient in need thereof. In one aspect, the sdAb comprises SBT-100 (SEQ ID NO:1).

In another embodiment, the invention includes a method for inhibiting disease caused by AMD and neovascular diseases of the eye, the method comprising administration of a sdAb to a patient in need thereof. In one aspect, the sdAb comprises SBT-100 (SEQ ID NO:1).

In another embodiment, the invention includes a method for inhibiting disease caused by VEGF, the method comprising administration of a sdAb to a patient in need thereof. In one aspect, the sdAb comprises SBT-100 (SEQ ID NO:1).

In another embodiment, the invention includes a method for down-regulation of PD-L1 expression, the method comprising administration of a sdAb to a patient in need thereof. In one aspect, the sdAb comprises SBT-100 (SEQ ID NO:1).

In another embodiment, the invention includes a method for inhibiting STAT3 translocation into the nuclei of cells, the method comprising administration of a sdAb to a patient in need thereof. In one aspect, the sdAb comprises SBT-100 (SEQ ID NO:1).

In another embodiment, the invention includes a method for inhibiting IL-6 effects, the method comprising administration of a sdAb to a patient in need thereof. In one aspect, the sdAb comprises SBT-100 (SEQ ID NO:1).

In another embodiment, the invention includes a method for potentiation of the efficacy of chemotherapeutic drugs the method comprising administration of a sdAb to a patient in need thereof. In one aspect, the sdAb comprises SBT-100 (SEQ ID NO:1). In another aspect, the chemotherapeutic drug comprises gemcitabine.

In another embodiment, the invention includes a method for penetrating the cell membrane, blood brain barrier, and blood retina barrier, the method comprising administration of a sdAb to a patient in need thereof. In one aspect, the sdAb comprises SBT-100 (SEQ ID NO:1).

In another embodiment, the invention includes a method for decreasing the toxicity of chemotherapeutic drugs, the method comprising administration of a sdAb to a patient in need thereof. In one aspect, the sdAb comprises SBT-100 (SEQ ID NO:1). In one aspect, the chemotherapeutic drug is doxorubicin. In another aspect, the sdAb comprises SBT-100 (SEQ ID NO:1).

In another embodiment, the invention includes a method for inhibiting the function of STAT3, the method comprising administration of a sdAb to a patient in need thereof. In one aspect, the sdAb comprises SBT-100 (SEQ ID NO:1).

In another embodiment, the invention includes a method for inhibiting KRAS and mutant KRAS function in cancer cells, the method comprising administration of a sdAb to a patient in need thereof. In one aspect, the sdAb comprises SBT-100 (SEQ ID NO:1).

In another embodiment, the invention includes a method for inhibiting VEGF production, the method comprising administration of a sdAb to a patient in need thereof. In one aspect, the sdAb comprises SBT-100 (SEQ ID NO:1).

In another embodiment, the invention includes a method for inhibiting PD-L1 expression, the method comprising administration of a sdAb to a patient in need thereof. In one aspect, the sdAb comprises SBT-100 (SEQ ID NO:1).

### DRAWINGS

These and other features, aspects, and advantages of the present invention will become better understood with regard to the following description, appended claims, and accompanying drawings where:

FIG. 1A-1D depicts SBT-100 (SEQ ID NO: 1) penetration of the cell membrane of MDA-MD-231 cells shown by immunofluorescence staining of MDA-MB-231 cells incubated with: FIG. 1A) SBT-100 (SEQ ID NO: 1) antibody, FIG. 1B) no VHH antibody FIG. 1C) negative control, and FIG. 0.1D) confocal image of SBT-100 (SEQ ID NO: 1) detection.

FIG. 2A-2F depicts fluorescent images showing intracellular levels of both phosphorylated and total STAT3 in MDA-MB-231 cells that were FIG. 2A) untreated, FIG. 2B) treated for 6 hour of with SBT-100 (SEQ ID NO: 1) treatment; confocal images of tSTAT3 in MDA-MB-231 cells that were FIG. 2C) untreated, or FIG. 2D) or following 6 hour of SBT-100 (SEQ ID NO: 1) treatment. Fluorescent images of pSTAT3 in MDA-MB-231 cells are shown, either FIG. 2E) untreated, FIG. 2F) or following 6 hour of SBT-100 (SEQ ID NO: 1) treatment. Confocal images of pSTAT3 in MDA-MB-231 cells are shown, either FIG. 2G) untreated, or FIG. 2H) following 6 hour of SBT-100 (SEQ ID NO: 1) treatment.

FIG. 3A depicts an immunoblot of MDA-MB-231 protein extracts for pSTAT3, tSTAT3, and PD-L1 with and without SBT-100 (SEQ ID NO: 1). FIG. 3B shows quantification of the immunoblot from FIG. 3A.

FIG. 4A-4F depicts a photograph showing fluorescent microscopy of phosphorylated STAT3 in Hep-2 cells under FIG. 4A) normal conditions, FIG. 4B) upon IL-6 stimulation, and with FIG. 4C) both IL-6 stimulation and SBT-100 (SEQ ID NO: 1) treatment; Panc-1 cells under FIG. 4D) normal conditions, FIG. 4E) upon IL-6 stimulation, and with FIG. 4F) both IL-6 stimulation and SBT-100 (SEQ ID NO: 1) treatment.

FIGS. 5A and 5B depicts KRAS GTPase activity measured by FIG. 5A) luminescence (RLU) and FIG. 5B) Western blot analysis for phosphorylated ERK1/2 in various KRAS mutant cancer cells with and without SBT-100 (SEQ ID NO: 1) treatment. Lanes 1 and 2 is MDA-MB-231 cells, Lane 3 and 4 is PANC-1 cells, and Lane 5 and 6 is BxPC3 cancer cells incubated with vehicle only.

FIG. 6 depicts a graph of tumor volume over time in athymic nude mice with and without treatment with SBT-100 (SEQ ID NO: 1).

FIG. 7A and FIG. 7B depicts photographs of immunohistochemistry of SBT-100 (SEQ ID NO: 1) in athymic nude mice with large established MDA-MB-231 tumors in FIG. 7A) tumor cells and FIG. 7B) brain cells.

FIG. 8 depicts fundoscopy scores for mice with and without SBT-100 (SEQ ID NO: 1) treatment.

FIG. 9A and FIG. 9B depicts the results from Optical Coherence Tomography with and without SBT-100 (SEQ ID NO: 1) treatment. Legend: GC/IPL: Ganglion Cell-Inner Plexiform Layer; OPL: Outer Plexiform Layer; INL: Inner Nuclear Layer; ONL: Outer Nuclear Layer; OLM: Ocular Larva Migrans; RPE/CC: Retinal Pigment Epithelium/Choriocapillaris.

FIG. 10A-10D depicts Electroretinogram (ERG) of EAU mice with and without SBT-100 (SEQ ID NO: 1) treatment.

FIG. 11A-11B depicts FIG. 11A) FACS analysis showing EAU mice with and without SBT-100 (SEQ ID NO: 1) treatment. FIG. 11B) Percentage of IFN-γ+IL-17A+ cells in EAU mice with and without SBT-100 (SEQ ID NO: 1) treatment.

FIG. 12 depicts immunoprecipitation studies in cells lines. M=Marker, lane 1=PANC-1 STAT3; lane 2=DU145; lane 3=HeLa+IFN-γ (P-STAT3); lane 4=4T1; lane 5=PANC-1 KRAS; lane 6=PC-3.

### DESCRIPTION

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

The term “antigenic determinant” refers to the epitope on the antigen recognized by the antigen-binding molecule (such as an sdAb or polypeptide of the invention) and more in particular by the antigen-binding site of the antigen-binding molecule. The terms “antigenic determinant” and “epitope” may also be used interchangeably. An amino acid sequence that can bind to, that has affinity for and/or that has specificity for a specific antigenic determinant, epitope, antigen or protein is said to be “against” or “directed against” the antigenic determinant, epitope, antigen or protein.

As used herein, the term “comprise” and variations of the term, such as “comprising” and “comprises,” are not intended to exclude other additives, components, integers or steps.

It is contemplated that the sdAbs, polypeptides and proteins described herein can contain so-called “conservative” amino acid substitutions, which can generally be described as amino acid substitutions in which an amino acid residue is replaced with another amino acid residue of similar chemical structure, and which has little or essentially no influence on the function, activity or other biological properties of the polypeptide. Conservative amino acid substitutions are well known in the art. Conservative substitutions are substitutions in which one amino acid within the following groups (a)-(e) is substituted by another amino acid within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp. Other conservative substitutions include: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

A “domain” as used herein generally refers to a globular region of an antibody chain, and in particular to a globular region of a heavy chain antibody, or to a polypeptide that essentially consists of such a globular region.

The amino acid sequence and structure of an sdAb is typically made up of four framework regions or “FRs,” which are referred to as “Framework region 1” or “FR1”; as “Framework region 2” or “FR2”; as “Framework region 3” or “FR3”; and as “Framework region 4” or “FR4,” respectively. The framework regions are interrupted by three complementarity determining regions or “CDRs,” which are referred as “Complementarity Determining Region 1” or “CDR1”; as “Complementarity Determining Region 2” or “CDR2”; and as “Complementarity Determining Region 3” or “CDR3,” respectively.

As used herein, the term “humanized sdAb” means an sdAb that has had one or more amino acid residues in the amino acid sequence of the naturally occurring VHH sequence replaced by one or more of the amino acid residues that occur at the corresponding position in a VH domain from a conventional 4-chain antibody from a human. This can be performed by methods that are well known in the art. For example, the FRs of the sdAbs can be replaced by human variable FRs.

As used herein, an “isolated” nucleic acid or amino acid has been separated from at least one other component with which it is usually associated, such as its source or medium, another nucleic acid, another protein/polypeptide, another biological component or macromolecule or contaminant, impurity or minor component.

The term “mammal” is defined as an individual belonging to the class Mammalia and includes, without limitation, humans, domestic and farm animals, and zoo, sports, and pet animals, such as cows, horses, sheep, dogs and cats.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, Ringer's solutions, dextrose solution, PBS (phosphate-buffered saline), and 5% human serum albumin. Liposomes, cationic lipids and non-aqueous vehicles such as fixed oils may also be used. The use of such media and agents for pharmaceutically active substances is well known in the art. Except insofar as any conventional media or agent is incompatible with a therapeutic agent as defined above, use thereof in the composition of the present invention is contemplated.

A “quantitative immunoassay” refers to any means of measuring an amount of antigen present in a sample by using an antibody. Methods for performing quantitative immunoassays include, but are not limited to, enzyme-linked immunosorbent assay (ELISA), specific analyte labeling and recapture assay (SALRA), liquid chromatography, mass spectrometry, fluorescence-activated cell sorting, and the like.

The term “solution” refers to a composition comprising a solvent and a solute, and includes true solutions and suspensions. Examples of solutions include a solid, liquid or gas dissolved in a liquid and particulates or micelles suspended in a liquid.

The term “specificity” refers to the number of different types of antigens or antigenic determinants to which a particular antigen-binding molecule or antigen-binding protein molecule can bind. The specificity of an antigen-binding protein can be determined based on affinity and/or avidity. The affinity, represented by the equilibrium constant for the dissociation of an antigen with an antigen-binding protein (KD), is a measure for the binding strength between an antigenic determinant and an antigen-binding site on the antigen-binding protein: the lesser the value of the KD, the stronger the binding strength between an antigenic determinant and the antigen-binding molecule (alternatively, the affinity can also be expressed as the affinity constant (KA), which is 1/KD). As will be clear to one of skill in the art, affinity can be determined depending on the specific antigen of interest. Avidity is the measure of the strength of binding between an antigen-binding molecule and the antigen. Avidity is related to both the affinity between an antigenic determinant and its antigen binding site on the antigen-binding molecule and the number of pertinent binding sites present on the antigen-binding molecule. Specific binding of an antigen-binding protein to an antigen or antigenic determinant can be determined by any known manner, such as, for example, Scatchard analysis and/or competitive binding assays, such as radioimmunoassays (RIA), enzyme immunoassays (EIA) and sandwich competition assays.

As used herein, the term “recombinant” refers to the use of genetic engineering methods (for example, cloning, and amplification) used to produce the sdAbs of the invention.

A “single domain antibody,” “sdAb” or “VHH” can be generally defined as a polypeptide or protein comprising an amino acid sequence that is comprised of four framework regions interrupted by three complementarity determining regions. This is represented as FR1-CDR1-FR2-CDR2-FR3-CDR3-FR4. An sdAb of the invention also includes a polypeptide or protein that comprises the sdAb amino acid sequence. Typically, sdAbs are produced in camelids such as llamas, but can also be synthetically generated using techniques that are well known in the art. As used herein, the variable domains present in naturally occurring heavy chain antibodies will also be referred to as “VHH domains,” in order to distinguish them from the heavy chain variable domains that are present in conventional 4-chain antibodies, referred to as “VH domains,” and from the light chain variable domains that are present in conventional 4-chain antibodies, referred to as “VL domains.” “VHH” and “sdAb” are used interchangeably herein. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113.

The term “synthetic” refers to production by in vitro chemical or enzymatic synthesis.

The term “target” as used herein refers to any component, antigen, or moiety that is recognized by the sdAb. The term “intracellular target” refers to any component, antigen, or moiety present inside a cell. A “transmembrane target” is a component, antigen, or moiety that is located within the cell membrane. An “extracellular target” refers to a component, antigen, or moiety that is located outside of the cell.

A “therapeutic composition” as used herein means a substance that is intended to have a therapeutic effect such as pharmaceutical compositions, genetic materials, biologics, and other substances. Genetic materials include substances intended to have a direct or indirect genetic therapeutic effect such as genetic vectors, genetic regulator elements, genetic structural elements, DNA, RNA and the like. Biologics include substances that are living matter or derived from living matter intended to have a therapeutic effect.

As used herein, the phrases “therapeutically effective amount” and “prophylactically effective amount” refer to an amount that provides a therapeutic benefit in the treatment, prevention, or management of a disease or an overt symptom of the disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The specific amount that is therapeutically effective can be readily determined by an ordinary medical practitioner, and may vary depending on factors known in the art, such as, e.g., the type of disease, the patient's history and age, the stage of disease, and the administration of other therapeutic agents.

STAT3 is an intracellular transcription factor that is activated by IL-6, other cytokines, and intracellular kinases, resulting in P-STAT3 turning on genes like vascular endothelial growth factor (VEGF) and promoting the differentiation of TH17 cells which are necessary for CNV and inflammatory diseases. Inhibiting STAT3 turns off VEGF production and prevents the generation of TH17 cells.

Despite the short half-life of VHHs in serum, camelid VHHs are increasingly considered for clinical use due to their ability to target antigens residing in tissues that are poorly vascularized and not easily accessible. In addition, VHHs are stable at room temperature and in reducing cytoplasmic environment. Described herein is a VHH, SBT-100 (SEQ ID NO: 1), with cell penetrating capability, that can bind to two different non-homologous intracellular targets (STAT3 and KRAS) implicated in tumorigenesis. SBT-100 (SEQ ID NO: 1) (1) crosses the cell membrane and binds to intracellular STAT3, (2) inhibits phosphorylation of STAT3, (3) decreases total STAT3, (4) blocks IL-6-mediated translocation of activated STAT3 into the nucleus, to prevent pSTAT3 dimers from binding to its target genes, (5) inhibits expression of vascular endothelial growth factor (VEGF), a key angiogenic factor and known modulator of tumor cells, (6) inhibits cell surface expression of check point inhibitor PD-L1 on tumor cell surface, which may improve antitumor immunity in immune-competent mice, (7) inhibits KRAS-GTPase activity and downstream ERK phosphorylation to inhibit cell growth, (8) exhibits wide-ranging anti-tumor cell growth in vitro against eleven human cancers, and (9) induces tumor (human cancers with activating KRAS mutations) regression in athymic xenograft mouse models for triple negative breast cancer cell line (MDA-MB-231) and for pancreatic cancer (PANC-1), without any observable toxicity. Biological effects of SBT-100 (SEQ ID NO: 1) are reversible, lasting at least 72 hours in vitro and 7 days in vivo. SBT-100 (SEQ ID NO: 1) also appears unique in its ability to penetrate BBB. The ability of SBT-100 (SEQ ID NO: 1) to penetrate the cell membrane and bind intracellular KRAS and STAT3 translates into functional suppression of cancer growth and proliferation in vitro and in vivo. This was demonstrated with multiple human cancers to show the broad application of SBT-100 (SEQ ID NO: 1) inhibiting human cancers.

The present invention relates to SBT-100 (SEQ ID NO: 1)'s capabilities to penetrate cell membranes and bind to STAT3 and with the ability to cross-react with KRAS (mutated and unmutated forms), acting as a bi-specific antibody with nM binding affinity. SBT-100 (SEQ ID NO: 1) can be administered therapeutically as an anti-cancer drug because it: (1) crosses the cell membrane and binds to intracellular STAT3, (2) inhibits phosphorylation of STAT3, (3) decreases total STAT3, (4) blocks IL-6-mediated translocation of activated STAT3 into the nucleus, which prevents p-STAT3 dimers from binding to its target genes, (5) inhibits expression of vascular endothelial growth factor (VEGF), a key angiogenic factor and known modulator of tumor cells, (6) inhibits cell surface expression of check point inhibitor PD-L1 on tumor cell surface, which may improve antitumor immunity in immune-competent mice, (7) inhibits KRAS-GTPase activity and downstream ERK phosphorylation, which inhibits cell growth, (8) exhibit wide-ranging anti-tumor cell growth in vitro, and (9) induce tumor regression in athymic xenograft mouse models for triple negative breast cancer cell line with KRAS (G13D) mutation (MDA-MB-231) and a pancreatic cancer cell line with KRAS (G12D) mutation (PANC-1), without any observable toxicity. Additionally, the biological effects of SBT-100 (SEQ ID NO: 1) last between 72 hours in vitro and 7 days in vivo.

The current standard of care in treating Age-Related Macular Degeneration (AMD) involves targeting VEGF and requires intraocular injections every 3 to 4 weeks. This anti-VEGF antibody therapy only targets one cytokine pathway—other cytokine pathways are not affected.

Higher levels of the systemic inflammatory markers CRP and IL-6 are independently associated with progression of AMD. Choroidal Neovascular (CNV) Membrane in AMD is associated with increased IL-6. Systemic IL-6 levels have been shown to correlate with the incidence and progression of AMD. IL-6 signaling may contribute to the pathogenesis of subretinal fibrogenesis in late-stage neovascular AMD.

Increased IL-10 in senescent eyes activates STAT3 signaling that induces activation of macrophages and vascular proliferation. Targeted inhibition of both IL-10 receptor mediated signaling and STAT3 activation in macrophages reverses the ageing phenotype.

IL-17 is involved in the pathogenic inflammation of AMD. IL-17 has a strong potential for stimulating neovascularization in a VEGF-independent manner. IL-17A reduces cellular viability, alters cell metabolism, and induces apoptosis in ARPE-19 cells. Aging and AMD-like degeneration are associated with increasing ocular IL-17 expression in mice.

Genetic deletion of SOCS3 in myeloid cells resulted in spontaneous STAT3 activation and accelerated CNV formation. Inhibition of STAT3 activation using a small peptide LLL12 suppressed laser induced CNV. STAT3 activation in circulating immune cells is related to neovascular age-related macular degeneration.

STAT3 is a transcription factor that transcribes VEGF, IL-6, IL-10, and IL-17. All these cytokines produce inflammatory changes in the retina that promote AMD. As such, inhibiting STAT3 will turn off the production of these AMD causing cytokines.

SBT-100 (SEQ ID NO: 1) internalizes into cells within 6 hours or less as demonstrated by immunohistochemical (IHC) staining. SBT-100 (SEQ ID NO: 1) inhibits VEGF protein production to near zero by human retinal epithelial cells in 12 hours or less. SBT-100 (SEQ ID NO: 1) inhibits PD-L1 cell surface expression by 10-fold within 24-48 hours. Both VEGF and PD-L1 are gene targets of the STAT3 transcription factor which is inhibited by SBT-100 (SEQ ID NO: 1).

SBT-100 (SEQ ID NO: 1) crosses the BBB in 15 minutes and can be stained within the neurons and glial cells of the mouse brain. Serum half-life of SBT-100 (SEQ ID NO: 1) in mice and rats is 1 hour. The biological half-life of SBT-100 (SEQ ID NO: 1) in a cancer xenograft model is 12-24 hours. The retinal biological half-life is at least 24-48 hours. SBT-100 (SEQ ID NO: 1) penetrates the cell membrane of 11 different types of human cancers and retinal cells, crosses the BBB, and also crosses the retinal blood barrier.

By blocking the inflammatory cascade and the vascular signaling pathways, inhibiting STAT3 may be effective in treating many ocular inflammatory and neovascular conditions such as corneal Neovascularization, proliferative diabetic retinopathy, keratoconjunctivitis sicca, AMD and uveitis. Corneal neovascularization is caused by a disruption of the balance between angiogenic and antiangiogenic factors that preserves corneal transparency. Proliferative Diabetic Retinopathy (PDR) mainly occurs when the blood vessels in the retina close, preventing blood flow. In an attempt to supply blood to the area where the original vessels closed, the retina responds by growing new blood vessels (neovascularization). These new blood vessels are abnormal and supply the retina with improper blood flow, and the new vessels are often accompanied by scar tissue which causes the retina to wrinkle or detach. Retinal ischemia promotes vasoproliferative factors that induce fibrous and neovascular growth which in turn leads to retinal damage. Keratoconjunctivitis sicca is the disruption of the ocular surface and tear film leads to inflammation and damage of the cornea. Is due to a chronic, bilateral desiccation of the conjunctiva and cornea due to an inadequate tear film (dryness). Subretinal neovascular Membrane from AMD-Choroidal neovascular membranes (CNVM) are new blood vessels that grow beneath the retina and disrupt vision. CNVM are associated with many serious eye diseases, most commonly ‘wet’ age-related macular degeneration (AMD).

Central nervous system (CNS) autoimmune diseases such as uveitis and multiple sclerosis result as consequence of breakdown of immune privilege of the brain, spinal cord or neuroretina which are maintained by the blood-retina barrier (BRB), blood-brain-barrier (BBB) and the neurovascular unit (NVU) comprised of pericytes, perivascular macrophages, tightly bound endothelial cells, glia limitans of the Miiller/microglia. These structures sequester CNS tissues from peripheral immune system and Th17 cells that produce Granzyme B are implicated in early events that initiate CNS autoimmune diseases by promoting the disruption of the BBB or BRB. However, sustained activation of microglial cells and recruitment of other inflammatory cells amplify the inflammatory response and are responsible for pathology characteristic of chronic uveitis or multiple sclerosis. Nonetheless, interventional studies using biologics such as cytokines or immune-suppressive compounds to suppress uveitis in mice invariably show strong correlation of disease amelioration with suppression of pathogenic Th17 cells. Subsequent studies revealed the requirement of STAT3 for Th17 differentiation and development while others showed that targeted deletion of STAT3 prevented the development of EAE or EAU. These studies led to the now established notion that targeting Th17 cells is a viable therapeutic approach for suppressing and mitigating autoimmune and autoinflammatory diseases.

Uveitis is a diverse group of potentially sight-threatening intraocular inflammatory diseases that is characterized by repeated cycles of remission and recurrent intraocular inflammation, and visual handicap is of significant public health importance as it affects patient's quality of life. Increased recruitment of Th17 cells into the retina is implicated in pathophysiology of uveitis and current therapies include periocular or intravitreal corticosteroid. However, their prolonged use for treatment of chronic uveitis is associated with development of serious side effects such as glaucoma and is the impetus for developing alternative therapies. Targeting the STAT3 pathway required for the differentiation and expansion of Th17 cells has been proposed as a potential therapy for mitigating uveitis because genetically modified mice that cannot induce Th17 cells are resistant to developing uveitis. However, a major impediment to targeting STAT3 pathway is that it is an intracellular protein and not accessible to STAT3-specific antibodies, as well as the unpredictable pharmacokinetic characteristics of small molecular weight STAT3 inhibitory peptides or mimetics.

Uveitis is ocular inflammation of the iris, ciliary body, or choroid that can occur from autoimmune conditions, trauma, and infections. A general term describing a group of inflammatory diseases that produces swelling and destroys the middle layer of tissues in the eye wall (uvea). The diseases includes sympathetic ophthalmia, birdshot retinochoroidopathy, Behcet's disease, Vogt-Koyanagi-Harada disease and ocular sarcoidosis. Is not limited to the uvea but also affects the lens, retina, optic nerve, and vitreous, producing reduced vision or blindness. Uveitis is a group of intraocular inflammatory diseases responsible for 10 percent of vision loss in the United States. Th17 T-helper cell subset has been implicated in the etiology of uveitis in mice and humans. STAT3 plays a critical role in the differentiation of Th17 cells and mice with targeted deletion of Th17 cells do not develop experimental autoimmune uveitis (EAU), the mouse model of human uveitis. Consequently, there is significant interest in developing drugs and biologics that target STAT3 pathway as therapy for uveitis and other inflammatory diseases.

SBT-100 (SEQ ID NO: 1) rapidly crosses the cell membrane in vitro in less than six hours, and in vivo it crosses the BBB in less than fifteen minutes. Upon entering the cell, SBT-100 (SEQ ID NO: 1) binds non-covalently to KRAS and STAT3 with nanomolar affinity. Unlike small molecule inhibitors which forms irreversible covalent bonds, SBT-100 (SEQ ID NO: 1) is less likely to create toxicity due to non-covalent reversible binding to KRAS and STAT3. The blocking of the GTPase activity of KRAS and subsequent decreases of the levels of pERK1/2 inhibits the ability of the KRAS pathway to promote cell proliferation, survival, and escape apoptosis. Concurrently, SBT-100 (SEQ ID NO: 1) also binds to STAT3 which causes inhibition of STAT3 phosphorylation, and the inability of STAT3 to translocate into the nucleus and prevent STAT3 from binding to its DNA promotor. A powerful example of SBT-100's (SEQ ID NO: 1) inhibitory and anti-inflammatory ability is also demonstrated by its ability to block the effect of IL-6 on cancer cells and normal cells in vitro by preventing STAT3 from transcribing target genes in the nucleus such as VEGF and PD-L1.

VEGF plays a critical role in tumor growth and metastasis by producing the development of new blood vessels. SBT-100 (SEQ ID NO: 1) significantly reduces the production of VEGF by retinal epithelial cells in vitro as rapidly as 12 hours, and the biological effect of a single administration lasts at least 48 hours. This suggests SBT-100 (SEQ ID NO: 1) may reduce anti-tumor effects in cancer and may reduce blindness in neovascular conditions such as age-related macular degeneration (AMD). In an in vivo model for blindness, SBT-100 (SEQ ID NO: 1) has been shown to give significant improvement in vision. Other gene targets for STAT3 are PD-1 and PD-L1. IFA demonstrates that SBT-100 (SEQ ID NO: 1) decreases PD-L1 expression on TNBC (MDA-MB-231) within 24 hours. Similar results were obtained for osteosarcoma (SJSA-1) where FACS analysis showed SBT-100 (SEQ ID NO: 1) decreased PD-L1 expression within 48 hours. This represents a new approach to immunotherapy by downregulating a checkpoint inhibitor gene. This strategy of using SBT-100 (SEQ ID NO: 1) may decrease the number of PD-L1 and possibly PD-1 molecules via decreasing STAT3 availability so there are fewer cell surface targets for nivolumab and pembrolizumab to block. This may augment the checkpoint inhibitor response or enable reductions in dosage of checkpoint inhibitors. Since STAT3 is a pro-inflammatory mediator, STAT3 inhibition by SBT-100 (SEQ ID NO: 1) may also decrease some of the inflammatory complications associated with checkpoint inhibitor therapy such as pneumonitis and severe COVID-19 pathology.

These results confirm the efficacy of SBT-100 (SEQ ID NO: 1) in the form of tumor regression an athymic nude mouse xenograft with TNBC tumors with KRAS(G13D) mutation (at least 50-100 mm³). The therapeutic effect of SBT-100 (SEQ ID NO: 1) persisted for at least seven days after the last dose. Similarly, SBT-100 (SEQ ID NO: 1) augmented suppression of tumor growth when combined with gemcitabine. PANC-1 is known to be a difficult to treat malignancy since it is KRAS-independent. These experiments suggest that SBT-100 (SEQ ID NO: 1) alone or in combination with other chemotherapeutic agents cause significant tumor growth suppression in vivo.

The most unique aspects of SBT-100 (SEQ ID NO: 1) described herein are intracellular penetrability and cross-reactivity with non-homologous KRAS. SBT-100 (SEQ ID NO: 1)'s novel property of penetrating the cell membrane and BBB gives it tremendous clinical potential in targeting diseases that are STAT3 mediated or cancers with KRAS mutations.

### EXAMPLES

### Example 1: Materials and Methods

SBT-100 (SEQ ID NO: 1) Development: Recombinant full-length human STAT3 with a GST tag fused to its N-terminal (STAT3-1496H) was provided by Creative BioMart (Shirley, NY). Briefly, a camel (Camelus bactrianus) was used for immunization with the recombinant human STAT3. Generating SBT-100 (SEQ ID NO: 1) VHH: A Camelid was immunized with the relevant antigen. After the immunization period, peripheral white blood cells (PWBC) were collected, and a phage display library was created to look for the VHH of interest. Once the panning process was completed, VHHs were identified by their binding affinity to STAT3 and KRAS. The final endotoxin levels were <1 EU/mg. The amino acid sequence of SBT-100 (SEQ ID NO: 1) (SEQ ID NO: 1) is: HVQLVESGGGSVQAGGSLRLSCAASGANGGRSCMGWFRQVPGKEREGVSGISTGGLIT YYADSVKGRFTISQDNTKNTLYLQMNSLKPEDTAMYYCATSRFDCYRGSWFNRYMYN SWGQGTQVTVSS). SBT-100 (SEQ ID NO: 1) has been previously described in U.S. patent Ser. No. 14/922,093, the contents of which are incorporated herein by reference.

Cell Lines and Cell Culture: Cell lines PANC-1, BxPC3, MDA-MB-231, MDA-468, MCF-7, BT474, U87, SJSA-1, HT-1080, HEp2, DU-145, and retinal epithelial cells (ARPE-19) were all obtained from American Type Culture Collection (ATCC) (Manassas, VA). All cells were grown at 37° C. in 5% CO₂ in either DMEM or RPMI media with or without fetal bovine serum.

Immunofluorescence and Immunohistochemical Staining: Standard procedures were used for immunohistochemistry and immunofluorescence assay (IFA) staining. Primary antibodies for IF were: Anti-t-STAT3: (Cell Signaling Technology), Anti-p-STAT3: (Cell Signaling Technology), Anti-PD-L1: (Cell Signaling Technology), Anti-VHH Antibody: (Rockland), Alexa Fluor 488-Anti-rabbitIgG: (AFanti-rabIgG, Jackson ImmunoResearch). The blocking solution, 1° and 2° antibody diluent were 1% BP: (1% BSA in PBS). All cell incubations were at 37° C. in a 5% CO₂ incubator in media. 4,500 cells/well were seeded in chamber slides allowed to adhere overnight. Cells were treated with SBT-100 (SEQ ID NO: 1) at various timepoints then fixed in 100% methanol at −20° C.

IFA: Wells were blocked for ≥30 minutes, blocking agent removed and primary antibodies were added at the following dilutions prior to overnight incubation at 5° C.: anti-VHH=1:500, anti-t-STAT3=1:300, anti-P-STAT3=1:125, anti-PD-L1=1:300. Wells were washed with PBS and incubated with AFanti-rabIgG (1:300) for ≥1 hour prior to washing with PBS, coverslipped and examined by fluorescent microscopy. For DAPI staining, wells were incubated for 7 minutes with 0.143 mM DAPI and washed with PBS prior to the application of a coverslip. Traditional fluorescence microscopy was performed using the Nikon 80i microscope and the appropriate wavelength filter. Images were captured using the attached Spot RT3 camera (model 25.4, 2 Mp slider) and the associated Spot 5.1 software.

Confocal microscopy: The confocal images were obtained using the Leica TCS SP8 confocal microscope. Images were quantified using Fiji software.

IHC Staining for Intra-tumor and BBB Methodology: Athymic nude mice (n=3) with established MDA-MB-231 tumors were injected IP with SBT-100 (SEQ ID NO: 1) (1 mg/kg). Fifteen minutes later the mice were sacrificed, and their brains and tumors were harvested. The tissues were placed into 10% formalin for 24 hours, and then transferred to 70% ethanol. The tissues were cut into sections with a dermatome (AML Laboratories, Baltimore, MD). Goat anti-llama conjugated (B ethyl Laboratories, Montgomery, TX) secondary antibody (1:10,000) was incubated with these tissue sections for 10 minutes at room temperature, washed twice for 3 minutes with PBS-Tween 20. Incubation in streptavidin/peroxidase complex of the tissue sections were done for 5 minutes at room temperature, and then washed for 5 minutes with PBS. Next the tissue sections were incubated with peroxidase substrate solution (AEC) for 15 minutes, washed in tap water for 5 minutes, counterstained with Hematoxylin QS (one drop on each section) and incubated for 30 seconds. Tissue sections were then rinsed with tap water until the water became colorless. The sections were mounted in aqueous mounting media, and 15 minutes later these slides were viewed on the Olympus BX51 Fluorescence Microscope.

Western Blot (Slot Blot): Standard procedures were used for immunoblotting. Primary antibodies were: Anti-Pactin: (Cell Signaling Technology), Anti-t-STAT3: (Cell Signaling Technology) Anti-P-STAT3: (Cell Signaling Technology), Anti-PD-L1: (Cell Signaling Technology), HRP-Anti-rabbit IgG: (Jackson ImmunoResearch). Blocking buffer, primary and secondary antibody diluent=5% BT: (5% BSA in TBS) TBS=Tris Buffered Saline: 25 mM Tris, 150 mM NaCl, pH 7.5 TBST=TBS+0.1% Tween-20. Briefly, 2×10⁵ MDA cells/well were seeded in each well of a 6-well plate and allowed to adhere overnight at 37° C. in a 5% CO₂ incubator in media. Media was removed and SBT-100 (SEQ ID NO: 1) in media was added. Following incubation for the indicated times, media was removed, the adherent monolayer of cells were washed with ice-cold PBS and then lysis of the cells was performed by scraping the cells in TBS+0.05% SDS with added EDTA, protease inhibitors and phosphatase inhibitors. At later times of treatment where non-adherent cells were apparent, these cells were collected by centrifugation, lysis was performed and combined with the lysate of the adherent cell population.

Quantification of Western Blot: The protein concentration of each fraction was determined using the BCA protein assay (ThermoFisher Scientific). Equal amounts of protein from each of the cell fractions (typically ˜10 μg/slot) was diluted to 200 μl/slot with TBS and loaded via a slot blot apparatus onto a PVDF membrane that had been previously activated in 100% methanol and then equilibrated in TBS. Blots were blocked for ≥1 hour in 5% BT and then incubated at 5° C. overnight in the following dilutions of antibodies: anti-βactin=1:1000, anti-t-STAT3=1:1000, anti-P-STAT3=1:750, anti-PD-L1=1:1000. Blots were washed three times with TBST, briefly equilibrated into TBS and then incubated with HRP-anti-rabIgG (1:5000) for ≥1 hour prior. The washing step was repeated and the blots incubated with the chemiluminescent HRP substrate according to the manufacturer's protocol. The reactions were visualized using a chemi-imager. Quantification was performed using the ImageJ software contained within the Fiji image processing package.

IL-6 stimulation and inhibition of p-STAT3 nuclear translocation: The cells (HEp-2 and PANC-1) were grown on 4 Permanox chambers slides. SBT-100 (SEQ ID NO: 1) antibody was added overnight (1 to 10 dilution in media) and the slides were kept at 37° C. No SBT-100 (SEQ ID NO: 1) antibody was added to the negative control samples. The following day, cells were stimulated with IL-6 (Peprotech, 100 ng/ml) for 15 minutes. After stimulation, the chamber slides were immediately fixed in ice cold 100% methanol for 10 minutes at 20° C. The slides were dried and proceeded with the previously mentioned IFA steps. Slides were blocked with 3% BSA in PBS at room temperature for 1 hour, then the primary antibody, Stat3 (Cell Signaling Technology) overnight at 4° C. The secondary antibody anti-mouse IgG (H & L), Alexa Fluor 488 (Cell Signaling Technology) was added for 1 hour at room temperature. Lastly, the chamber slides were washed and mounted with mounting media and viewed in the Nikon Fluorescence microscope.

Promega Dual Luciferase Reporter Assay System (GTPase-Glo™ Assay): In this assay, a HEK 293 IL-6 STAT3 reporter cell line (Promega, Madison WI) was used to measure STAT3 transcriptional activity. In this cell line, induction with 40 ng/ml of IL-6 activates STAT3 transcription factors to drive luciferase reporter expression which can then be measured on a standard luminometer. 10⁵ cells/well were incubated with SBT-100 (SEQ ID NO: 1) antibody or no antibody for 48 hours. An 8 point, 2-fold titration, starting at 100 ug/ml was made in order to test the IC50 values. IL-6 was added during the last 18 hours of incubation. All time points are clocked from the addition of SBT-100 (SEQ ID NO: 1) inhibitor. At 48 hours, cells underwent lysis and luminescence measurements were made using a BMG Labtech microplate reader. Results are expressed as a percent of control wells (cells+IL-6).

Human VEGF-A ELISA Assay: The human VEGF-A ELISA (ThermoFisher Scientific) assay was modified to be performed in a 96-well plate format from a 24-well format. ARPE-19 cells were serum starved in 10% FBS in DMEM and incubated overnight. The media was then replaced with media containing SBT-100 (SEQ ID NO: 1) at 100, 10, 1 or 0.1 μg/ml, Anti-EMP2 antibody (Abcam), or just media and incubated for 12, 24 or 48 hours. At the appropriate time point, the supernatant was removed and stored at ≤−65° C. Cells were lysed using RIPA lysis buffer (ThermoFisher Scientific) and the protein content of the cell lysate was measured with BCA assay (ThermoFisher Scientific). VEGF-A was measured utilizing the Human VEGF-A ELISA Kit (ThermoFisher Scientific) in accordance with the kit instructions. Experimental statistical analysis by ANOVA with Dunnett Multiple Comparisons Test using the negative control as the control column was performed. Statistical analysis by ANOVA with Dunnett Multiple Comparison Test using the negative control as the control was performed.

Flow Cytometry Analysis: SJSA-1 cells were incubated for 24 hours with 50 ng/ml recombinant human IFN-γ (Peprotech), then media was replaced with media containing 50 μg/ml of SBT-100 (SEQ ID NO: 1) and incubated for 48 hours. Cells were then harvested and stained with the following antibodies: CD276 (Clone MIH-42, Biolegend), CD274 (Clone 29E.2A3, Bilegend), and CD200 (Clone OX-104, Bilegend). Upon staining, samples were run through a BD Celesta Flow Cytometer and data were analyzed using FlowJo software.

MTT Assay: For these experiments, cancer cells were grown until they reached a confluency of 90%. Cells were washed, trypsinized and counted using a Coulter Counter (Beckman, Brea, CA). The proliferation studies were carried out using the 3-[4,5-dimethylthialolyl]-2,5-diphenyl-tetrazolium bromide (MTT) assay (Roche Diagnostics Corporation, Cat. No. 11465007001, Sigma-Aldrich). For this, cells were seeded in a 96-well plate at a density of 5×10³. Cells were allowed to adhere for 24 hours and treated at the appropriate concentrations (serial dilutions beginning at 100 ug/ml) as described in Table 2. At day 3, 10 ul of MTT reagent (0.5 mg/ml) was added to each well as indicated by manufacturer. After a 4-hour incubation period, 100 ul of solubilization solution was added and the plate was placed in the incubator overnight. All the plates were read at 570 nm wavelength using the Biotek plate reader (Winooski, VT). All data were analyzed using GraphPad InStat3 (GraphPad Software, Inc., La Jolla, CA). Treatment groups were compared with vehicle control group using one-way ANOVA. If a significant difference (p<0.05) was observed, then the Tukey-Kramer multiple comparison test was conducted.

Measurement of KRAS Inhibition Activity in an Enzymatic Assay: The GTPase activity of KRAS converts GTP to GDP. A GTPase-Glo reagent kit (Promega, Madison WI) is designed to measure this activity. The Glo reagent converts unhydrolyzed GTP to ATP and yields a luminescent signal. When KRAS activity is inhibited, GTP remains unhydrolyzed and a high Luminescent signal is expected. If KRAS is not inhibited, then the GTP is converted to GDP and a low signal is observed. Luminescence was measured using a PHERAstar plate reader (BMG Labtech). The activity of mature, active KRAS (SignalChem, Richmond, BC Canada) supplied in the manufacturer's buffer was tested against the presence of a dilution series of inhibitors. The commercial KRAS GTPase activity was titrated in Promega GTPase/GAP buffer and in SBT-100 (SEQ ID NO: 1) buffer in presence of several inhibitors. The inhibition and the effect of buffer conditions for the GTPase activity of KRAS was compared.

Animals: All animals were housed under pathogen-free conditions and experiments were performed in accordance with Illinois Institute of Technology (ITT) Research Institute Animal Use and Care Committee (IACUC) which is accredited by the Association for Assessment and Accreditation of Laboratory Animal Care International (AAALAC). Athymic nude-Foxn1nu female mice aged 5 to 6 weeks were purchased from ENVIGO Laboratories (Indianapolis, IN). Animals were quarantined for one week and housed five mice per cage, with a 12-hour light-dark cycle, at 20° C.-26° C., and a relative humidity of 50%. Drinking water and diet (PicoLab Rodent Diet 20 Irradiated consisting of 20% crude protein, 4.5% crude fat, and 6.0% crude fiber) were supplied to the animals ad libitum.

Murine Xenograft Models: Tumor cells in passage five were used for the implantation and were harvested during log phase growth. PANC-1 cells or MDA-MB-231 cells at a concentration of 5×10⁶ cells per 100 μl of media were injected subcutaneously into the right flank. Tumor measurements were initiated as soon as the tumors were palpable. Thereafter, tumors were measured twice weekly. Tumors were measured in two dimensions using calipers and volume was calculated using the formula: Tumor volume (mm³)=(w²×1)/2; where w=width and 1=length in mm of a tumor. Animals were randomized using the stratified random sampling algorithm when tumors reached a size range of 79-172 mm³ for PANC-1 tumors and 55-150 mm³ for the MDA-MB-231 tumors. Treatment of the animals with SBT-100 (SEQ ID NO: 1) or vehicle injected via intraperitoneal route was initiated the day following randomization referred to as day 1. Study Log Study Director Animal Study Management Software (San Francisco, CA) was used to randomize animals, collect data (e.g., dosing, body weights, tumor measurements, clinical observations), and conduct data statistical analyses.

### Example 2: Binding Characteristics of Anti-STAT3 and Anti-KRAS Camelid VHH: SBT-100 (SEQ ID NO: 1)

Recombinant full-length human STAT3 with a GST tag fused to its N-terminal (STAT3-1496H) was provided by Creative BioMart (Shirley, NY, USA). Briefly, a camel (Camelus bactrianus) was used for immunization with the recombinant human STAT3. After the immunization protocol was completed, peripheral blood cells were collected for total RNA isolation and two rounds of nested PCRs to create a VHH library. Next, clones were selected that best exhibited cell penetration and binding to human STAT3 and human KRAS proteins. In this screening, high affinity binding of SBT-100 (SEQ ID NO: 1) to recombinant STAT3 and KRAS, both wild type and mutated form (G12D), was observed using Biacore 3000 (Table 1). For scouting, the sample was allowed to flow over the chip, and the binding of sample to the ligand was monitored in real time. The affinity constant (KD) was determined as a ratio of dissociation and association rates. SBT-100 (SEQ ID NO: 1) binds wild type KRAS with a KD=4.20×10⁻⁹, KRAS(G12D) with KD=1.50×10⁻⁸, and STAT3 with KD=2.24×10⁻⁸. SBT-100 (SEQ ID NO: 1) does not bind an irrelevant antigen (12-Lipoxygenase). Although STAT3 and KRAS molecules are highly conserved in nature, they do not share significant homology in their protein sequences. While binding to its immunogen STAT3 was expected, the cross-reactivity to KRAS was not. The sequence of the anti-KRAS VHH (SBT-102) (SEQ ID NO. 2) used was EVQLVESGGGSVQTGGSLRLSCAVSGNIGSSYCMGWFRQAPGKKREAVARIVRDGATG YADYVKGRFTISRDSAKNTLYLQMNRLIPEDTAIYYCAADLPPGCLTQAIWNFGYRGQG TLVTVSS. Thus, SBT-100 (SEQ ID NO: 1) can bind to and inhibit cancers with wild type KRAS, KRAS(G12D) (most common mutant), and KRAS(G13D) mutations. SBT-100 (SEQ ID NO: 1) likely binds a common epitope near the KRAS GTPase active site, thereby making SBT-100 (SEQ ID NO: 1) a pan-KRAS inhibitor. The bi-specific binding of SBT-100 (SEQ ID NO: 1) sdAb to KRAS and STAT3 may allow it to concentrate inside cells with high concentration of STAT3 which then brings SBT-100 (SEQ ID NO: 1) into close proximity to KRAS.

Table 1 also shows the binding of anti-Ebola VP24 VHH (SBT-106) (SEQ ID NO. 3: EVQLVESGGGSVQAGGSLRLSCAASVYSYNTNCMGWFRQAPGKEREGVAVIYAAGGL TYYADSVKGRFTISQENGKNTVYLTMNRLKPEDTAMYYCAAKRWCSSWNRGEEYNY WGQGTQVTVSS) and anti-HIV-1 Reverse Transcriptase (RT) VHH (SBT-107) (SEQ ID NO. 4): MGDVQLVESGGDSVRAGGSLQLSCKASGYTYNSRVDIRSMGWFRQYPGKEREGVA TINIRNSVTYYADSVKGRFTISQDNAKNTVYLQMNALKPEDTAMYYCALSDRFAAQVP ARYGIRPSDYNYWGEGTLVTVSSSSGLE) to Ebola VP24 and HIV-1 RT, respectively.

TABLE 1
Binding affinity of SBT-100 for human STAT3 and KRAS determined using Biacore 3000

| | | | Human | Human | | |
|---|---|---|---|---|---|---|
| | Human | Human | KRAS | KRAS | | |
| | STAT3 | STAT5 | Wildtype | (G12D) | Ebola | HIV-1 |
| | Protein | Protein | protein | Mutant | VP24 | RT |
| Anti-KRAS/Anti- | 2.24 × 10⁻⁸ | 7/50 × 10⁻⁸ | 4.20 × 10⁻⁹ | 1.50 × 10⁻⁸ | | |
| STAT3 VHH | | | | | | |
| (SBT-100 (SEQ | | | | | | |
| ID NO: 1)) | | | | | | |
| Anti-KRAS VHH | No Binding | No Binding | 3.22 × 10⁻⁹ | 1.48 × 10⁻⁷ | | |
| (SBT-102 (SEQ | Observed | Observed | | | | |
| ID NO: 2) | | | | | | |
| Ebola VP24 VHH | | | | | 2.94 × 10⁻⁸ | |
| (SBT-106 (SEQ | | | | | | |
| ID NO: 3) | | | | | | |
| HIV-1 RT VHH | | | | | | 3.85 × 10⁻⁹ |
| (SBT-107 (SEQ | | | | | | |
| ID NO: 4) | | | | | | |

### Example 3: Cellular Penetration by SBT-100 (SEQ ID NO: 1)

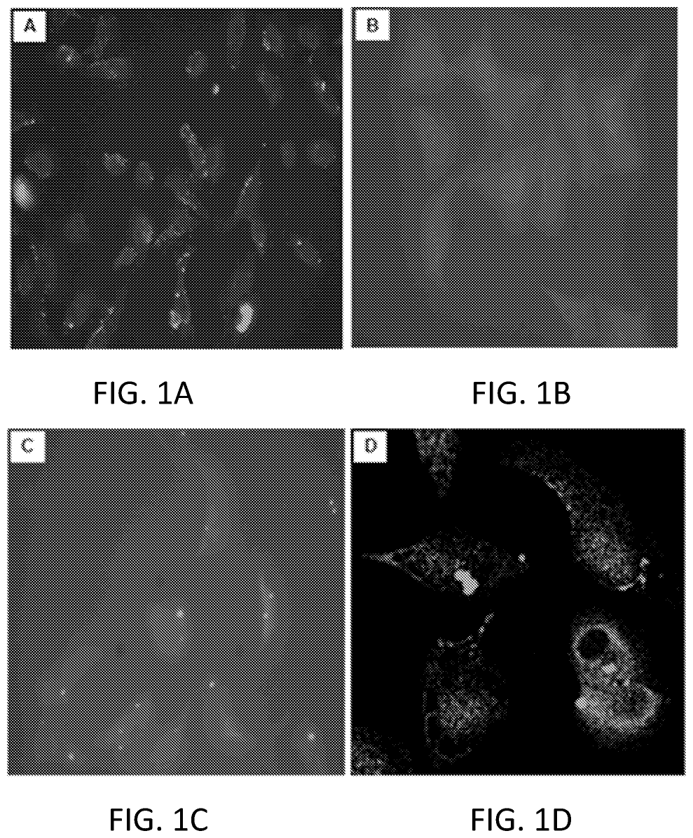

The ability of SBT-100 (SEQ ID NO: 1) VHH to penetrate the cell membrane and bind intracellular STAT3 was determined using immunofluorescence analyses (IFA) of MDA-MD-231 TNBC cell line. Cells treated with SBT-100 (SEQ ID NO: 1) exhibited intracellular localization of SBT-100 (SEQ ID NO: 1) as well as some membrane association was shown using anti-GST tag antibody in the TNBC cell line, MDA-MD-231. FIG. 1A shows immunofluorescence staining of MDA-MB-231 cells incubated with SBT-100 (SEQ ID NO: 1) antibody with positive cytoplasmic staining using the anti-His tag antibody in the green channel. FIG. 1B shows the negative control of MDA-MB-231 cells treated with vehicle only. As expected, the cells treated with vehicle showed no staining. As an additional negative control, MDA-MB-231 cells were cultured with an anti-HIV-1 reverse transcriptase VHH (FIG. 1C); because the MDA-MB-231 cells do not produce HIV-1 viruses, no intracellular staining was noted in these cells with traditional fluorescence microscopy. FIG. 1D depicts a confocal image of SBT-100 (SEQ ID NO: 1) detection via an anti-VHH antibody, showing granular staining located throughout the cytoplasm of the MDA-MB-231 cells. Confocal fluorescent microscopy revealed that SBT-100 (SEQ ID NO: 1) is present as granular staining dispersed throughout the cytoplasm of MDA-MB-231 cells following treatment with SBT-100 (SEQ ID NO: 1) (FIG. 1D). The observed staining of SBT-100 (SEQ ID NO: 1) in FIG. 1A and FIG. 1D is cytosolic, not endocytic.

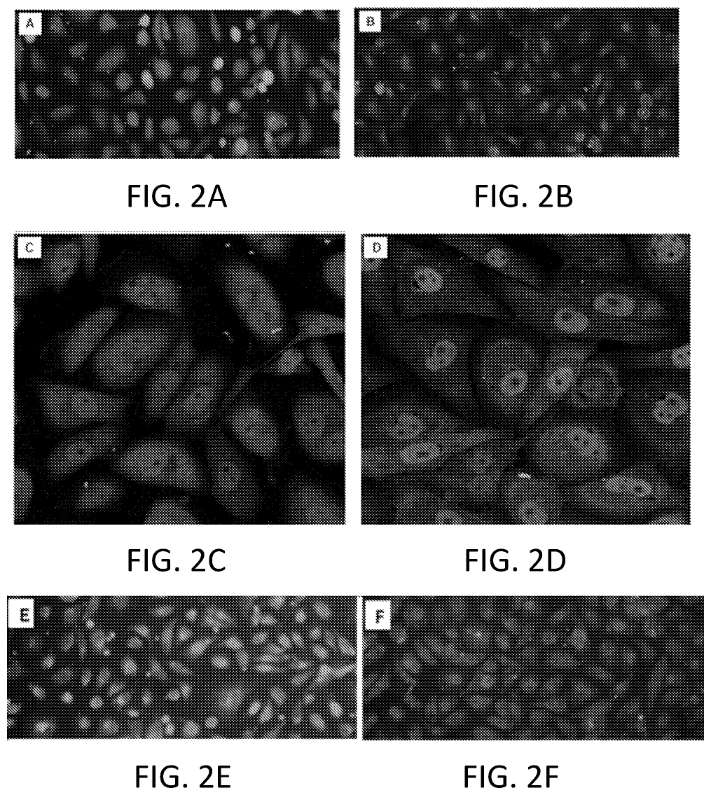

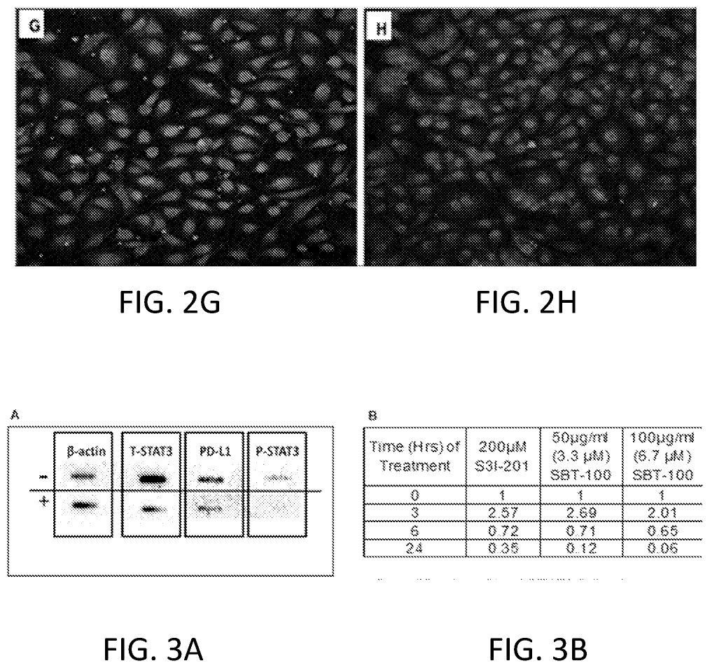

The effect of SBT-100 (SEQ ID NO: 1) on intracellular levels of STAT3 was determined. As shown in FIG. 2A and FIG. 2B, IFA shows that SBT-100 (SEQ ID NO: 1) reduces intracellular levels of both phosphorylated and total (t) STAT3. MDA-MB-231 cells were either untreated (FIG. 2A) or treated for 6 hours with 150 μg/ml SBT-100 (SEQ ID NO: 1) (FIG. 2B). There was a redistribution of tSTAT3 from predominantly nuclear and around the nucleus to tSTAT3 dispersed throughout the cell, indicating that nuclear localization t-STAT3 is reduced in SBT-100 (SEQ ID NO: 1)-treated MDA-MB-231 cells. Confocal images of tSTAT3 in MDA-MB-231 cells, either untreated (FIG. 2C) or following 6 hours of 150 μg/ml SBT-100 (SEQ ID NO: 1) treatment (FIG. 2D) demonstrates that treatment with SBT-100 (SEQ ID NO: 1) decreases intracellular levels of phosphorylated (p) STAT3 in MDA-MB-231 cells. Additionally, confocal fluorescent image analysis for both tSTAT3 and DAPI (4′,6-diamidino-2-phenylindole)-stained nuclei further supports this SBT-100 (SEQ ID NO: 1)-induced redistribution of tSTAT3 staining from nuclear/cell-centralized to staining throughout the whole cell cytoplasm (FIG. 2C and FIG. 2D). Protein sequence analysis using online tool “Prot Pi” (https://www.protpi.ch/Calculator/ProteinTool#Results) provided a theoretical net charge of only +2.34 at physiological pH, deviating substantially from suggested optimal net charge of +14. Thus, SBT-100 (SEQ ID NO: 1) appears unique in its ability to penetrate the cell, bind non-homologous proteins (STAT3 and KRAS) and cross the blood brain barrier (BBB). Cell penetration by VHH has been shown by others; however, this was only seen following cationic resurfacing of the molecules. The ability of SBT-100 (SEQ ID NO: 1) to penetrate the cell membrane does not involve poly-cationic resurfacing, genetic engineering or conjugation of monomers.

### Example 4: Inhibition of Phosphorylation and Nuclear Translocation of STAT3 with Subsequent Reduction in Check Point Molecules

Following the demonstration of cell penetrating capacity of SBT-100 (SEQ ID NO: 1), the effects on pSTAT3 in vitro were evaluated. IFA indicated that SBT-100 (SEQ ID NO: 1) decreased intracellular levels of tSTAT3. Furthermore, there was a redistribution of tSTAT3 from predominantly nuclear and around the nucleus to tSTAT3 dispersed throughout the cell, indicating that nuclear localization tSTAT3 is reduced in SBT-100 (SEQ ID NO: 1)-treated MDA-MB-231 cells. Confocal fluorescent image analysis for both tSTAT3 and DAPI-stained nuclei further supports this SBT-100 (SEQ ID NO: 1)-induced redistribution of tSTAT3 staining from nuclear/cell-centralized to staining throughout the whole cell cytoplasm (FIGS. 2C and 2D). IFA studies demonstrated the MDA-MB-231 cells incubated with SBT-100 (SEQ ID NO: 1) demonstrated decreased pSTAT3 levels within 6 hours. Microscopy was done on MDA-MB-231 cells that were untreated (FIG. 2E and FIG. 2G) or following 6 hours of 150 μg/ml SBT-100 (SEQ ID NO: 1) treatment (FIG. 2F and FIG. 2H). FIGS. 2E, 2F, 2G, and 2H show reduced levels of pSTAT3 after treatment. SBT-100 (SEQ ID NO: 1)-induced changes in pSTAT3 cellular localization mirrored those of tSTAT3. These data suggest that SBT-100 (SEQ ID NO: 1) hinders the translocation of both total and phosphorylated STAT3 from the cytoplasm to the nucleus. Thus, both conventional and confocal fluorescent images revealed that SBT-100 (SEQ ID NO: 1) decreased intracellular levels of pSTAT3 in MDA-MB-231 cells.

The effect of SBT-100 (SEQ ID NO: 1) on immune checkpoint inhibitors in cancer cells, such as PD-L1, a transcriptional gene target of STAT3 was investigated. Checkpoint molecules are expressed on the surface of tumor cells and ultimately stifle the functioning of activated of T cells, contributing to the immunosuppressive tumor microenvironment. Inflammatory cytokines such as IL-6 and IFN-γ drive the expression of checkpoint molecules such as PD-L1 (CD274), B7-H3 (CD276), and OX-2 (CD200). Culturing the human osteosarcoma cell line SJSA-1 with SBT-100 (SEQ ID NO: 1) resulted in decreased cell surface expression of B7-H3, OX-2, and PD-L1 upon stimulation with 75 ug/ml IFN-γ in SJSA-1 osteosarcoma cells in the presence or absence of SBT-100 (SEQ ID NO: 1) (data not shown). B7-H3 decreased by approximately 3-fold, OX-2 decreased by approximately 4-fold, and PD-L1 decreased by approximately 4-fold. MDA-MB-231 cells were analyzed after 24 hours of treatment with SBT-100 (SEQ ID NO: 1) to examine potential changes in the level of PD-L1 protein synthesis within other cell types. Fluorescent microscopy of PD-L1 expression was shown in MDA-MB-231 cells that were stimulated for 24 hours with 75 ug/ml IFN-γ, either without SBT-100 (SEQ ID NO: 1) and with SBT-100 (SEQ ID NO: 1) (data not shown). Confocal fluorescent microscopy of PD-L1 expression in MDA-MB-231 cells is shown upon 24 hour stimulation with 75 ug/ml IFN-γ, either with or without the addition of SBT-100 (SEQ ID NO: 1) (data not shown). PD-L1 staining using IFA indicated lower membrane and intracellular protein levels mediated by SBT-100 (SEQ ID NO: 1), consistent with published data on the expression of cellular PD-L1 localization. Interferon (IFN)-γ is known to induce PD-L1 expression and both the intracellular and membrane staining increased following treatment with IFN-γ (data not shown). This is the first demonstration of downregulation of checkpoint-inhibitors at the gene expression level by an antibody.

The changes in the protein levels were quantitated using immunoblot technique. FIG. 3A shows a representative image of immunoblotting of MDA-MB-231 protein extracts for pSTAT3, tSTAT3, and PD-L1 with and without SBT-100 (SEQ ID NO: 1) at 24 hours. The changes in the levels of tSTAT3, pSTAT3 and PD-L1 proteins induced by SBT-100 (SEQ ID NO: 1) treatment of MDA-MB-231 cells were quantified by immunoblotting of protein extracts from untreated MDA-MB-231 cells and cells treated for 3 hours, 6 hours, and 24 hours with SBT-100 (SEQ ID NO: 1) using S31-201, a STAT3 inhibitor as a positive control (FIG. 3B and data not shown). Each data point represents the average±the standard deviation of the β-actin normalized values from 3-5 independent experiments where MDA-MB-231 cells were treated with 4 different SBT-100 (SEQ ID NO: 1) preparations whose final concentration varied between 50 μg/ml-1-100 μg/ml. Replicate immunoblots were reacted with antibodies for each of the three proteins under investigation. Following chemiluminescent detection, the signal intensity of each protein in each extract was normalized to the intensity of the β-actin present in that sample. The levels in each treated sample were then compared to the level found in untreated cells (set to 1) to quantify the differences in the levels of tSTAT3, pSTAT3 and PD-L1 induced at multiple times post SBT-100 (SEQ ID NO: 1) administration. S3I-201, a known STAT3 phosphorylation inhibitor which has been shown to decrease levels of PD-L1, was used as a positive control. SBT-100 (SEQ ID NO: 1) treatment reduced levels of tSTAT3, p-STAT3 and PD-L1, with the most significant reduction of all three proteins at 24 hours. Thus, quantification confirmed the data observed by immune fluorescence.

The nuclear portion of the staining with the commercially purchased tSTAT3 antibody and the pSTAT3 antibody were determined from the two-dimensional images by quantification of the green antibody-specific fluorescence that co-occurred with the nuclear (blue DAPI) staining. Following 6-hour treatment with SBT-100 (SEQ ID NO: 1), 41% of the original tSTAT3 staining and 70% of the original pSTAT3 staining remained in the nucleus. Of the tSTAT3 still present, none remained within the nucleus based on a three-dimensional projection of the corresponding Z-stacked images obtained by confocal microscopy. Any tSTAT3 that co-stained with DAPI in the two-dimensional images was present in the cytoplasm surrounding the nuclear membrane. The very low staining intensity of the pSTAT3 antibody in the IFA system makes this analysis impossible using that antibody. These data show that SBT-100 (SEQ ID NO: 1), like S3I-201, decreased tSTAT3, pSTAT3 and PD-L1 over the course of the 24-hour treatment. There appeared to be an early (3 hour) increase in all three-protein species prior to a decrease. The reduction in tSTAT3 was evident at 6-hour and preceded that of pSTAT3, while reduced PD-L1 levels were apparent 24 hours after treatment with SBT-100 (SEQ ID NO: 1). Taken together, these data support the hypothesis that binding of STAT-3 to SBT-100 (SEQ ID NO: 1) decreases PD-L1 translation by decreasing levels of the pSTAT3 transcription factor.

### Example 5: Inhibition of IL-6 Induced STAT3 Nuclear Translocation and Suppression of STAT3 Mediated VEGF Production

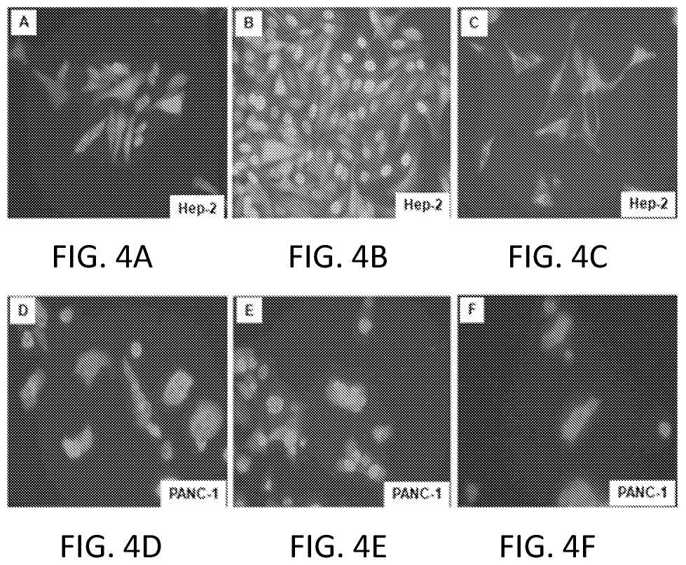

Since IL-6 plays a key role in modulating nuclear translocation of activated pSTAT3, experiments were done to determine whether SBT-100 (SEQ ID NO: 1) mediates its activity by inhibiting IL-6 activity. As shown in FIG. 4, IL-6 stimulation of HEp-2 and PANC-1 cells with SBT-100 (SEQ ID NO: 1) resulted in inhibited nuclear translocation of pSTAT3, subsequently reducing proliferation of HEp-2 and PANC-1 cells. Fluorescent microscopy of phosphorylated STAT3 in Hep-2 cells is shown under normal conditions (FIG. 4A), upon IL-6 stimulation (FIG. 4B), and with both IL-6 stimulation and SBT-100 (SEQ ID NO: 1) treatment (FIG. 4C). Fluorescent microscopy of phosphorylated STAT3 in Panc-1 cells is shown under normal conditions (FIG. 4D), upon IL-6 stimulation (FIG. 4E), and with both IL-6 stimulation and SBT-100 (SEQ ID NO: 1) treatment (FIG. 4F). Reporter cell assays were done to measure luciferase expression from HEK 293 cells transfected with a construct linking the STAT3 promoter to the luciferase gene. STAT3 luciferase reporter assay in HEK 293T cells with SBT-100 (SEQ ID NO: 1) treatment after IL-6 stimulation shows that STAT3 dimers translocate to the nucleus and bind to the STAT3 promoter, inducing luciferase activity. Addition of SBT-100 (SEQ ID NO: 1) abrogated this effect in a dose dependent manner. Thus, the IL-6 effect on STAT3 activity was significantly inhibited by SBT-100 (SEQ ID NO: 1). This assay showed that treatment with SBT-100 (SEQ ID NO: 1) renders near 100% reduction in IL-6 induced pSTAT3 binding to its DNA promotor at a dose of 100 μg/mL, with an IC₅₀ of 2.68 μg/mL (0.18 μM) (data not shown).

At 12, 24, and 48-hour time points, SBT-100 (SEQ ID NO: 1) at 100 ug/ml gave >99% suppression of VEGF protein production (p<0.01)

In cancer cells, STAT3 transcribes many genes necessary for the growth and survival of the cells, such as VEGF. To determine the ability of SBT-100 (SEQ ID NO: 1) to inhibit VEGF production, retinal epithelial cells were used that produce VEGF in large quantities. VEGF levels were measured by ELISA in retinal epithelial cells stimulated with IL-6 in the presence of increasing concentrations of SBT-100 (SEQ ID NO: 1). At 12, 24, and 48-hour time points, SBT-100 (SEQ ID NO: 1) at 100 μg/mL gave >99% suppression of VEGF protein production (data not shown). No cell toxicity was observed at any concentration tested in this experiment (data not shown). VEGF is a critical cytokine for the growth and survival of cancer cells, and it is well known that inhibition of VEGF function can significantly improve overall survival of cancer patients (colorectal, lung, glioblastoma, kidney, cervical, and ovarian cancers). For instance, bevacizumab hinders the effect of VEGF by binding it in the extracellular space. SBT-100 (SEQ ID NO: 1) penetrates the cell membrane and inhibits STAT3 function resulting in significantly decreased VEGF protein production. This is a completely unique way of inhibiting VEGF as compared to bevacizumab.

There are high levels of IL-6 in the blood of patients with severe infection with COVID-19. IL-6 plays a key role in the tumor microenvironment of many cancers and promotes STAT3 mediated inflammation in the eye causing macular degeneration and uveitis. SBT-100 (SEQ ID NO: 1) may reduce SARS-CoV-2 replication in patients by binding and inhibiting STAT3. By blocking IL-6 effects, SBT-100 (SEQ ID NO: 1) may help reduce pulmonary inflammation which may then improve the patient's pulmonary compliance and oxygenation.

### Example 6: SBT-100 (SEQ ID NO: 1) Inhibits Growth of Human Cancers with KRAS Mutations and Constitutive Expression of pSTAT3 In Vitro

SBT-100 (SEQ ID NO: 1) impaired growth in eleven human cell lines derived from a variety of cancers, including pancreatic cancers (PANC-1 and BxPC3), TNBCs (MDA-MB-231, MDA-MB-468, MDA-MB-453), ER+PR+ breast cancer (MCF-7), HER-2+ amplified breast cancer (BT474), glioblastoma (U87), osteosarcoma (SJSA-1), fibrosarcoma (HT-1080), and metastatic, chemo-resistant prostate cancer (DU-145) (Table 2). The MDA-MB-231 cells have a KRAS(G13D) and PANC-1 cells have a KRAS(G12D) activating mutation. Most of these cancer cells have constitutive expression of pSTAT3. These results demonstrate that SBT-100 (SEQ ID NO: 1) possesses a wide spectrum of anti-cancer activity. This data suggests that SBT-100 (SEQ ID NO: 1) has significant (p<0.001) tumor cell inhibitory effects (85-93%) against human malignancies with constitutive pSTAT3 expression with or without an activating KRAS mutation.

TABLE 2
SBT-100 (SEQ ID NO: 1) inhibited in vitro growth of eleven tumor cell lines

| | | | % Inhibition in 3 | |
|---|---|---|---|---|
| | | KRAS | days with SBT-100 | |
| | Human Cancer | Mutation/ | (SEQ ID NO: 1) | |
| Cancer | Cell Line | STAT3 | ug/ml or 6.7 uM) | IC50 (μM) |
| Pancreatic | PANC-1 | +/+ | 85% (P < 0.001) | 0.38 |
| Pancreatic | Bx-PC3 | −/+ | 90% (P < 0.001) | 2.34 |
| TNBC | MDA-MB-231 | +/+ | 89% (P < 0.001) | 1.21 |
| TNBC | MDA-MB-468 | ?/+ | 85% (P < 0.001) | 0.83 |
| TNBC | MDA-M-453 | −/+ | 64% (P < 0.001) | 1.19 |
| Breast | MCF-7 | ?/+ | 93% (P < 0.001) | 0.99 |
| Breast | BT474 | ?/+ | 93% (P < 0.001) | 1.68 |
| Glioblastoma | U87 | −/+ | 62% (P < 0.001) | 4.32 |
| Osteosarcoma | SJSA | ?/+ | 83% (P < 0.001) | 3.39 |
| Fibrosarcoma | HT-1080 | −(+NRAS)/+ | 86% (P < 0.001) | 2.13 |
| Prostrate | DU-145 | +/+ | 92% (P < 0.001) | 1.46 |

### Example 7: Inhibition of KRAS GTPase Activity and Suppression of Downstream pERK Signaling

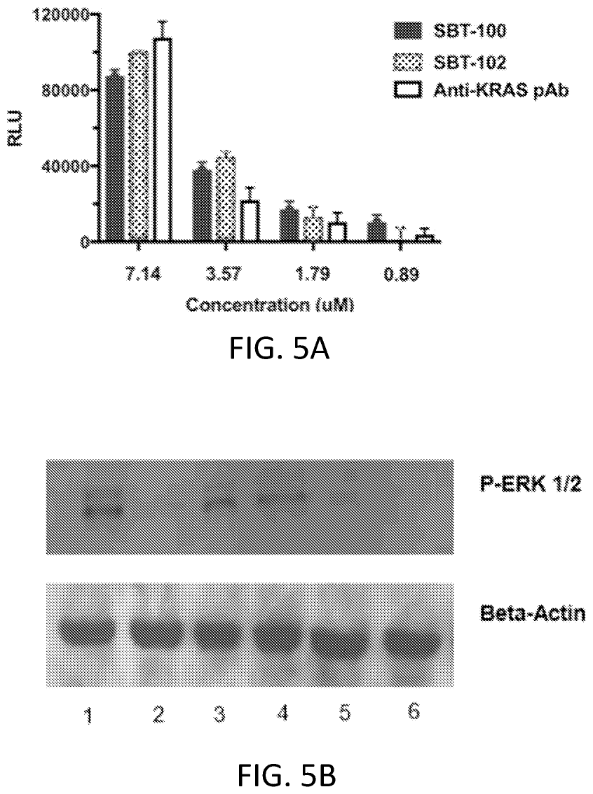

Binding data demonstrates SBT-100 (SEQ ID NO: 1) is bi-specific for KRAS and STAT3 as determined using these proteins and Lipoxygenase, while SBT-102 is mono-specific for human KRAS and its most common mutant KRAS(G12D) with nanomolar affinity but does not bind STAT3 (Table 1). The biochemical effect of SBT-100 (SEQ ID NO: 1) and SBT-102 (SEQ ID NO: 2) on KRAS was demonstrated using GTPase activity. Luminescence (RLU) was measured as a readout for KRAS GTPase activity. Reagents were incubated with either SBT-100 (SEQ ID NO: 1), SBT-102 (SEQ ID NO: 2), or anti-KRAS polyclonal antibody and RLU were measured. KRAS GTPase activity was inhibited by increasing doses of SBT-100 (SEQ ID NO: 1) and SBT-102 (SEQ ID NO: 2), demonstrating its inhibitory binding activity (FIG. 5A). Here, anti-KRAS polyclonal antibody was used as a positive control. Inhibition of KRAS GTPase activity by SBT-100 (SEQ ID NO: 1), SBT-102 (SEQ ID NO: 2), and anti-KRAS polyclonal antibody occurred in a dose dependent manner, and the results between the three were comparable. Western blot analysis was performed for phosphorylated ERK1/2 in various KRAS mutant cancer cells with and without SBT-100 (SEQ ID NO: 1) treatment (FIG. 5B). In Lane 1 (MDA-MB-231), Lane 3 (PANC-1), and Lane 5 (BxPC3) cancer cells were incubated with vehicle only. In Lane 2 (MDA-MB-231), Lane 4 (PANC-1), and Lane 6 (BxPC3) cancer cells were incubated with 100 ug/ml of SBT-100 (SEQ ID NO: 1) for 72 hours. The results show downstream phosphorylation of ERK (pERK) is inhibited in both MDA-MD-231 and PANC-1 cancer cell lines harboring KRAS mutations. By binding KRAS and inhibiting its GTPase activity, SBT-100 (SEQ ID NO: 1) blocks the KRAS signaling pathway. It is possible that cancer cells with greater basal STAT3 activity will trap SBT-100 (SEQ ID NO: 1) within the cytoplasm to a greater extent, thereby enhancing SBT-100's (SEQ ID NO: 1) ability to inhibit KRAS. This is likely the explanation for the difference in pERK levels between these cancer cell lines. It has been demonstrated that MDA-MB-231 has much higher levels of STAT3 than PANC-1. This may result in decreased time for SBT-100 (SEQ ID NO: 1) to bind and inhibit KRAS. In addition, the literature shows that PANC-1 is KRAS-independent whereas other cancers with KRAS mutations are KRAS-dependent. This may also explain the lower reductions in pERK levels in PANC-1 versus MDA-MB-231. Thus, SBT-100 (SEQ ID NO: 1) binds to KRAS, subsequently, inhibition of KRAS GTPase activity and suppression of downstream KRAS signaling reduces pERK levels which results in the inhibition of the growth of cancer cells with activating KRAS mutations.

### Example 8: Reduction in Tumor Volume with SBT-100 (SEQ ID NO: 1) in Tumor Bearing Mice

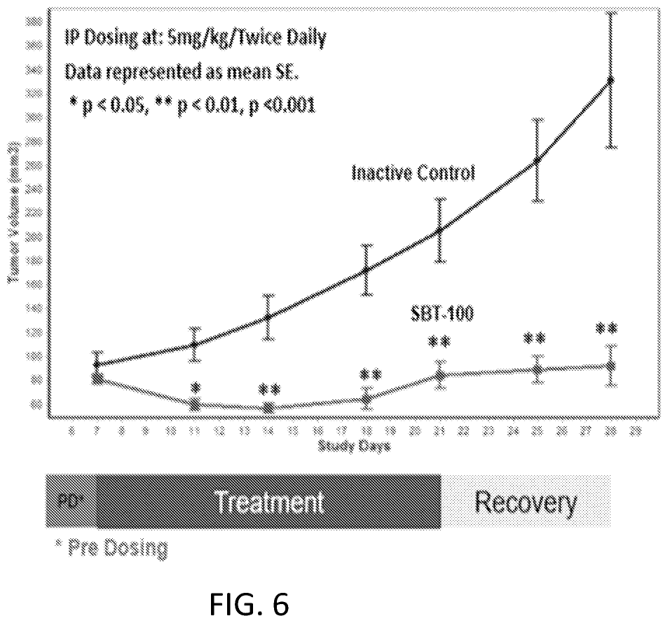

Athymic mice subcutaneously injected with TNBC cell line (MDA-MB-231) or pancreatic cancer cells (PANC-1) were treated for 14 days with SBT-100 (SEQ ID NO: 1) and allowed to recover for 7 days, followed by measurement of tumor volume. MDA-MB-231 tumors were between 50-100 mm³ prior to initiation of treatment (FIG. 6). The tumor-bearing mice were randomized into a control group which received PBS and a treatment group that received 5 mg/kg SBT-100 (SEQ ID NO: 1) (BID) via intraperitoneal injection for 14 days until sacrifice. The treatment group demonstrated rapid suppression of tumor growth, and this was maintained even after the 14-day treatment period. During the 7-day post-treatment observation period no rebound in tumor growth occurred, and all the mice survived treatment and maintained their normal weight. After the last dose of SBT-100 (SEQ ID NO: 1), there was no significant growth of the MDA-MB-231 during the observed period.

PANC-1 tumors were between 100-150 mm³ prior to treatment and then randomized into four groups: control (PBS), gemcitabine only (20 mg/kg, once daily), SBT-100 (SEQ ID NO: 1) only (100 mg/kg, BID), and gemcitabine with SBT-100 (SEQ ID NO: 1), all via intraperitoneal injection for 14 days. After the 14-day period of treatment, there was a 7-day period of observation. At the end of the study, the gemcitabine only group had 14.93% tumor growth suppression versus the control group (Table 3). The SBT-100 (SEQ ID NO: 1) only group had 19.17% tumor growth suppression versus the control group. Finally, the combination group of gemcitabine with SBT-100 (SEQ ID NO: 1) showed 31.52% suppression versus the control group (p<0.05). No treated mice died or had any weight loss with SBT-100 (SEQ ID NO: 1). To determine the potential toxicity of SBT-100 (SEQ ID NO: 1), the weight data from all xenograft studies where the mice received SBT-100 (SEQ ID NO: 1) were combined. During the 3-week xenograft studies there was no significant weight loss in the groups receiving SBT-100 (SEQ ID NO: 1) for treatment (data not shown). Reasons for the lack of observable toxicity may include the short serum half-life while biological effects lasting for up to 7-days which are reversible. In addition, the role of STAT3 in normal adult tissues is limited. Conditional ablation of STAT3 in adult mice has been shown to impact different systems to varying degrees without being lethal, unlike embryonic targeting that result in lethality. It may also be possible that due to aberrant levels of intracellular STAT3 expression in cancer cells, the injected SBT-100 (SEQ ID NO: 1) may be preferentially accumulating in the cancer lesions.

TABLE 3
% Inhibition of Control

| Group | Day 12 | Day 15 | Day 19 | Day 22 |
|---|---|---|---|---|
| 20 mg/kg Gemcitabine | 19.51 | 23.17 | 23.26 | 14.93 |
| 5 mg/kg SBT-100 (SEQ ID NO: 1) BID | 20.7 | 27.29 | 18.37 | 19.17 |
| 5 mg/kg SBT-100 (SEQ ID NO: 1) | 28.83 | 35.48 | 36.37 | 31.52 |
| BID + 20 mg/kg Gemcitabine | | | | |

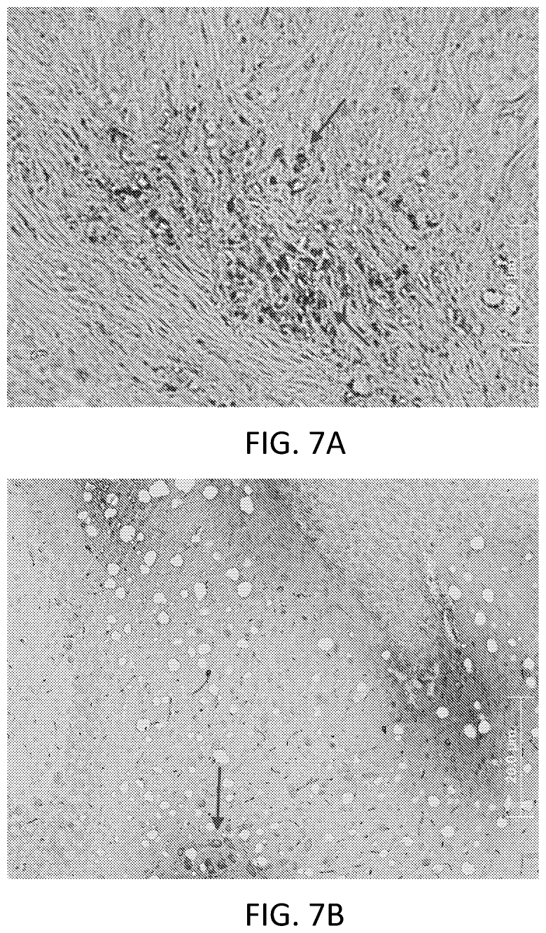

Another important finding in vivo was that SBT-100 (SEQ ID NO: 1) crosses the blood brain barrier (BBB). Athymic nude mice with large established MDA-MB-231 tumors (>200 mm³) were injected once IP with 5 mg/kg of SBT-100 (SEQ ID NO: 1) for fifteen minutes and then sacrificed. Immunohistochemistry analysis demonstrates localization of SBT-100 (SEQ ID NO: 1) inside cancer cells across the blood brain barrier (BBB). (FIG. 7A) This representative section of the tumor mass shows intracellular staining within the TNBC cells as indicated by the arrows. These cells are surrounded by dense tumorstroma. (FIG. 7B) The arrow points to a collection of intracellular staining of neurons and glial cells within the brains of the tumor bearing mice. It has been demonstrated that VHHs with basic pI (9.3 but not 7.7) could spontaneously cross BBB. SBT-100 (SEQ ID NO: 1) has a basic pI of 8.22 (calculated using Prot Pi online tool), which is outside the reported pI that enables BBB crossing.

### Example 9: Ophthalmic Disease

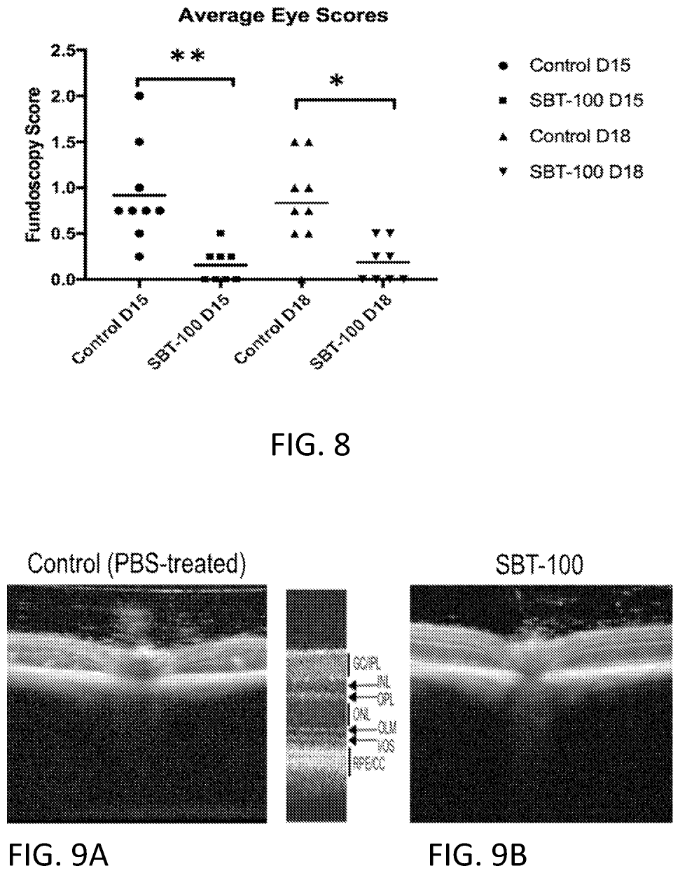

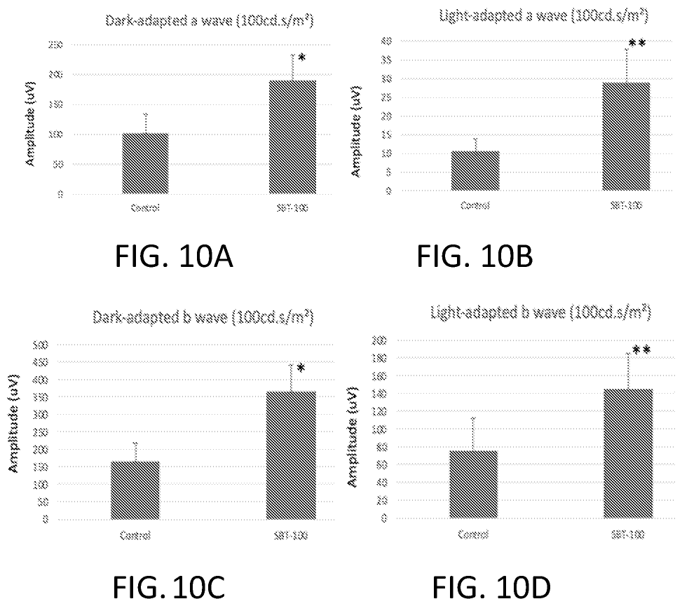

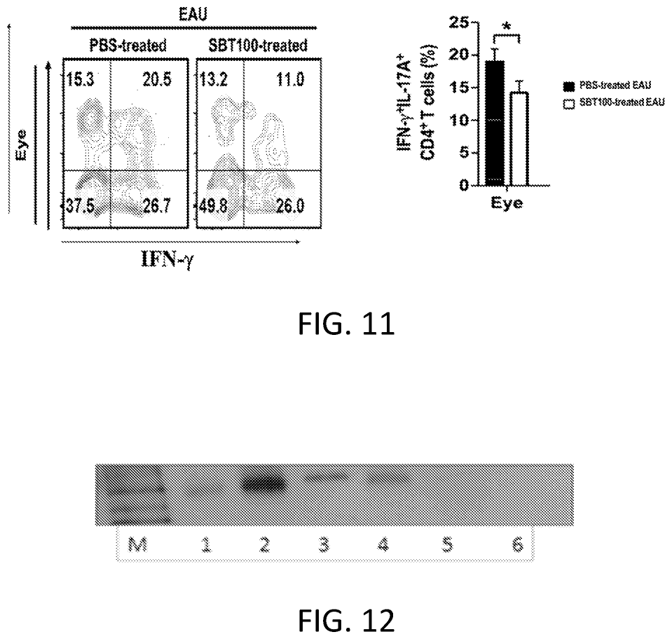

Experimental Autoimmune Uveitis (EAU) was induced in mice. To do this, mice were immunized with interphotoreceptor retinoid-binding protein (IRBP) in an emulsion of CFA and pertussis toxin. Once EAU was induced, SBT100 was then injected into the eye. The effects of SBT-100 (SEQ ID NO: 1) on uveitis were examined by fundoscopy on day 15 and 18, Optical Coherence Tomography (OCT) on day 15, and Electroretinography (ERG) on day 14. Fundoscopy is an exam that uses a magnifying lens and a light to check the fundus of the eye (back of the inside of the eye, including the retina and optic nerve). Mice were sacrificed on day 18, the eyes were harvested for histology and intracellular cytokine analysis was done by FACS. As shown in FIG. 8, EAU clinical scores are decreased in SBT-100 (SEQ ID NO: 1) treated mice as measured by fundoscopy. The fundoscopy images showed that SBT-100 (SEQ ID NO: 1) ameliorated uveitis (data not shown). The OCT results showed reduced recruitment of inflammatory cells at the optic nerve head and vitreous (FIGS. 9A and 9B). FIG. 10A-D depicts the ERG results showing improved visual function in the SBT-100 (SEQ ID NO: 1) treated EAU mice. FIG. 11 shows that treatment with SBT-100 (SEQ ID NO: 1) intracellular cytokine staining analysis results in significant reduction of IL-17 secreting pathogenic Th17 cells. Thus, SBT-100 (SEQ ID NO: 1) attenuated the severity of uveitis in a mouse model of human uveitis, suggesting that the single domain SBT-100 (SEQ ID NO: 1) can be used as therapy for Uveitis and other inflammatory diseases.

### Example 10: SBT-100 (SEQ ID NO: 1) Binds STAT3 from Lysed Cancer Cell Lines

It has been previously shown that SPT-100 can bind unphosphorylated STAT3. FIG. 12 demonstrates the binding of phosphorylated STAT3 and SBT-100 (SEQ ID NO: 1). Cell lysates were incubated for 1 hour at 4° C. with Dynabeads preloaded with SBT-100 (SEQ ID NO: 1), positive control (commercial STAT3), or negative control (commercial STAT1). Bound proteins were separated by SDS-PAGE followed by Western blot analysis using STAT3 (79D7) rabbit mAb #4904. FIG. 12 shows the amount of STAT3 cells: lane 1 is PANC-1 cells, lane 2 is DU145 cells, lane 3 is HeLa cells, lane 4 is 4T1 cells with mouse STAT3 (which is 99% homologous to human STAT3), lane 5 is a negative control with PANC-1 and KRAS and lane 6 is PC-3 cells. The immunoblot shows that PANC-1 cells have low intracellular STAT3, DU145 prostate cancer cells have high concentration of STAT3. This may explain why STAT3 is more effectively suppressed compared to other cells with less STAT3.

### Example 11: STAT3 in Viral Infections

Infection with Ebola virus impairs the host's immune response and causes a cytokine storm. The Ebola virus VP24 protein binds karyopherin alpha1 and blocks STAT1 nuclear accumulation. The Ebola virus interferon antagonist VP24 directly binds STAT1. In the absence of STAT1, no antiviral state is established. STAT3 signaling is activated by DNA and RNA viruses, including EBV, MCMV, HCMV, HSV, VZV, and HCV. Activation or increased expression of STAT3 is required for the replication of a number of viruses by suppressing the type I IFN mediated antiviral response or regulating microtubule dynamics. STAT3 inhibitors decrease viral replication significantly.

Table 4 shows that SBT-100 (SEQ ID NO: 1) inhibits Ebola virus proliferation in Hela cells, as well as in HFF cells. Additionally, SBT-100 (SEQ ID NO: 1) inhibits Zika virus (DAKAR, Senegal) proliferation in Vero cells and HFF cells. SBT-100 (SEQ ID NO: 1) inhibits Venezuelan Equine Encephalitis (TC83) virus proliferation in different cell lines. SBT-100 (SEQ ID NO: 1) inhibits Chikungunya virus proliferation.

TABLE 4
Viral inhibition by SBT-100 (SEQ ID NO: 1)

| | Cell | | Max % | EC50 | |
|---|---|---|---|---|---|
| SBT sdAb | Line | Pathogen | Inhibition | [μM] | Comment |
| SBT-100 | HeLa | ENBOV | 97% | 1.27 | Inhibits Ebola virus replication |
| (SEQ ID NO: 1) | | (Ebola) | | | comparable to control compounds |
| SBT-106 | HeLa | ENBOV | 45% | 3.49 | |
| (SEQ ID NO: 3) | | (Ebola) | | | |
| SBT-100 | HFF | ENBOV | 95% | 2.56 | Inhibits Ebola virus replication |
| (SEQ ID NO: 1) | | (Ebola) | | | comparable to control compounds |
| SBT-106 | HFF | ENBOV | No | No | Does not inhibit Ebola virus |
| (SEQ ID NO: 3) | | (Ebola) | response | response | replication in this model |
| SBT-100 | Vero | Zika | 96% | 0.74 | Inhibits Ebola virus replication |
| (SEQ ID NO: 1) | | | | | comparable to control compounds |
| SBT-106 | Vero | Zika | No | No | Does not inhibit Ebola virus |
| (SEQ ID NO: 3) | | | response | response | replication in this model |
| SBT-100 | HeLa | TC83 | 96% | 0.665 | Inhibits Venezuelan Equine |
| (SEQ ID NO: 1) | | | | | Encephalitis (VEE) virus |
| | | | | | proliferation |
| SBT-100 | BE3M27 | TC83 | 97% | 5.94 | Inhibits Venezuelan Equine |
| (SEQ ID NO: 1) | | | | | Encephalitis (VEE) virus |
| | | | | | proliferation |
| SBT-100 | U87MG | TC83 | 96% | 2.53 | Inhibits Venezuelan Equine |
| (SEQ ID NO: 1) | | | | | Encephalitis (VEE) virus |
| | | | | | proliferation |
| SBT-100 | U205 | CHIV | 99% | 5.05 | Inhibits Chickungunya (CHIV |
| (SEQ ID NO: 1) | | (181/25) | | | 181/25) virus proliferation |
| SBT-100 | Vero | Zika DAKAR, | 98% | 5.45 | Inhibits Zika virus replication |
| (SEQ ID NO: 1) | | Senegal | | | |
| SBT-100 | HFF | Zika DAKAR, | 98% | 6.26 | Inhibits Zika virus replication |
| (SEQ ID NO: 1) | | Senegal | | | |

SBT-100 (SEQ ID NO: 1) may inhibit the replication of other viruses such as: hemorrhagic fever viruses including, Dengue, Marburg, Arenaviruses (Lassa & Junin Viruses), Bunyaviruses; Toga Viruses (Alpha Viruses) such as Mosquito-borne Encephalitis Viruses, West Nile Virus (WNV), Venezuelan Equine Encephalitis Virus (VEE), Eastern Equine Encephalitis Virus (EEE), Western Equine Encephalitis Virus (WEE); Chikungunya Virus, and Coronaviruses including Severe Acute Respiratory Syndrome (SARS) and Middle East Respiratory Syndrome (MERS).

As shown above, SBT-100 (SEQ ID NO: 1) inhibits IL-6. IL-6 is a key mediator of inflammation and uses the STAT3 pathway to do this. There is high levels of IL-6 in the blood of patients with severe infection with COVID-19. IL-6 plays a key role in the tumor microenvironment of many cancers and promotes STAT3 mediated inflammation in the eye causing macular degeneration and uveitis. SBT-100 (SEQ ID NO: 1) may reduce SARS-CoV-2 replication in patients by binding and inhibiting STAT3. By blocking IL-6 effects, SBT-100 (SEQ ID NO: 1) may help reduce pulmonary inflammation which may then improve the patient's pulmonary compliance, and oxygenation.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claim should not be limited to the description of preferred embodiments contained in this disclosure. All references cited herein are incorporated by reference in their entirety.

Insofar as the description above discloses any additional subject matter that is not within the scope of the claims below, the inventions are not dedicated to the public and the right to file one or more applications to claim such additional inventions is reserved.

What is claimed is:

1. A method of preventing aberrant cell proliferation in a subject using a single-domain antibody (sdAb) directed against an intracellular component.

2. The method of claim 2, wherein the aberrant cell proliferation is cancer.

3. The method of claim 2, wherein the cancer is selected from the group comprising osteosarcoma, fibrosarcoma, glioblastoma, leukemia, pancreatic cancer, breast cancer, and prostate cancer.

4. The method of claim 2, wherein the sdAb is synergistic with one or more chemotherapeutic drugs and improves therapeutic efficacy of the one or more chemotherapeutic drug against cancer.

5. The method of claim 4, wherein the one or more chemotherapeutic drugs comprises doxorubicin.

6. The method of claim 4, wherein the one or more chemotherapeutic drugs comprises gemcitabine.

7. The method of claim 4, wherein the sbAb decreases the toxicity of one or more chemotherapeutic drugs and improves survival in the treated subject.

8. The method of claim 1, wherein the sbAb is used in combination with one or more compounds.

9. The method of claim 1, wherein the intracellular component comprises a protein.

10. The method of claim 8, wherein the protein comprises STAT1, STAT2, STAT3, STAT4, STAT5a, STAT5b, or STAT6.

11. The method of claim 1, wherein the sdAb comprises SEQ ID NO:1.
